(19) United States
(12) Patent Application Publication
(10) Pub. No.: US 2023/0166156 A1
(43) Pub. Date: Jun. 1, 2023

(54) SUSPENSION TRAINING SYSTEMS WITH MACHINE LEARNING CAPABILITIES

(71) Applicant: Hygear Ltd., Tel Aviv (IL)

(72) Inventors: Guy Bar, Tel Aviv (IL); Eyal David, Kfar Saba (IL); Elad Elkalai, Tzur Yitzhak (IL); Idan Hail, Yaad (IL); Paul M. Juris, Amherst, MA (US); Elisha Poplinger, Kiryat Ono (IL); Dan Strik, Los Angeles, CA (US)

(21) Appl. No.: 18/046,145

(22) Filed: Oct. 12, 2022

Related U.S. Application Data
(60) Provisional application No. 63/254,996, filed on Oct. 12, 2021.

(51) Int. Cl. A63B 24/00; A63B 21/00

(52) U.S. Cl. CPC A63B 24/0062; A63B 21/151; A63B 21/4035; A63B 2220/51; A63B 2225/02; A63B 2071/065

(57) ABSTRACT

Suspension training systems include one or more bands, and one or more handles. The handles can be coupled to the bands, and transmit user-generated forces to the bands. The systems also include a force sensor that measures the user-generated forces acting on the bands, and a computing device that acquires and displays information relating to the exercise session. The systems can generate recommendations for future exercise sessions based on the acquired information, so that exercise sessions can be tailed to the specific needs and capabilities of the user.

### CROSS-REFERENCE TO RELATED APPLICATIONS

This application claims the benefit under 35 U.S.C. § 119(e) of U.S. Provisional Application No. 63/254,996 filed Oct. 21, 2021, the contents of which are incorporated by reference herein their entirety.

### BACKGROUND

Suspension training systems typically include one or more flexible, inelastic bands, and a handle connected to each of the bands. The bands can be securely anchored to a wall or other stationary structure. As the user pulls the handles away from the anchoring structure, the bands assume a state of tension. The anchoring structure, in turn, exerts a reactive force that the user feels as a resistance to the pulling of the handles. The resistive force causes one or more muscle groups within the user to become activated, exercising those muscle groups. The user can target a specific muscle group, and can vary the resistive force, by orienting the user's body in a particular manner in relation to the anchoring point, and by exerting force on the handles in a particular direction.

The user can perform active and static exercises using the suspension training system. During active exercises, the user moves one or more body parts, usually in a repetitive manner, and varies the force that the user exerts on the handles. During static exercises, the user does not move, or attempts to not move, while the user maintains, or attempts to maintain, a substantially constant force on the handles.

The optimum exercise routine can vary widely between users, depending on factors such as the height, weight, strength, and fitness level of the user. Also, a user may wish to target a particular muscle group or body part with an exercise routine; and the user may desire to obtain a certain result from an exercise program, such as strengthening, toning, weight loss, etc. And the optimum exercise routine for a particular user can vary over time as the fitness level of the user increases or decreases. Thus, it can be difficult for a user to develop and perform exercise routines that optimally and consistently satisfy the unique needs and goals of the user.

Also, while it can be highly beneficial for a user to perform structured, predetermined exercise routines tailored to that particular user, it can be challenging for the user to achieve, maintain, and track the various exercise metrics such as force, repetitions, timing, etc. throughout the routine, and to properly adjust the routine while it is being performed if the level of difficulty is too low, or too high.

Also, suspension training systems, in general, do not have real time feedback capabilities that present to the user the resisting force, mechanical work, number of repetitions, and other valuable data for managing the workout and boosting training experience. It is believed that interactive real time data, such as the resistive force, mechanical work performed by the user, calories burned, etc., can encourage or boost the user into engaging and meeting workout goals, and can lead the user to a better exercise experience in general.

### SUMMARY

In one aspect of the disclosed technology, an exercise system includes at least one band; at least one handle configured to be coupled to the at least one band; and a force sensor. The force sensor includes a load cell having a beam configured to be coupled to the at least one band, and to an anchoring point. The load cell also includes at least one strain gauge mounted on the beam. The force sensor also includes a first computing device communicatively coupled to the strain gauge and having a processor configured to determine a force acting on the force sensor based on an output of the strain gauge. The system further includes a second computing device communicatively coupled the first computing device and configured to display information relating to an exercise session performed on the system by a user.

In another aspect of the disclosed technology, the beam is a substantially S-shaped beam having a first arm configured to be coupled to the at least one band; and a second arm configured to be coupled to the anchoring point.

In another aspect of the disclosed technology, the force sensor further includes a first strap coupled to the first arm of the beam and configured to be coupled to the at least one band; and a second strap coupled to the second arm and configured to be coupled to the anchoring point.

In another aspect of the disclosed technology, the first strap has a first loop formed therein; the first strap is connected to the first arm of the beam by way of the first loop; the second strap has a second loop formed therein; and the second strap is connected to the second arm of the beam by way of the second loop.

In another aspect of the disclosed technology, the at least one band is an inelastic band.

In another aspect of the disclosed technology, the beam of the load cell further includes a first lip located at a freestanding end of the first arm, and a second lip located at a freestanding end of the second arm. The first lip is configured to retain the first strap on the first beam; and the second lip is configured to retain the second strap on the second beam.

In another aspect of the disclosed technology, a distance between the first lip and a non-freestanding end of the first arm is about equal to a width of the first strap; and a distance between the second lip and a non-freestanding end of the second arm is about equal to a width of the second strap.

In another aspect of the disclosed technology, the first computing device includes a memory having calibration data for the load cell stored therein; and the processor is further configured to determine the force acting on the force sensor based on the output of the strain gauge and the calibration data.

In another aspect of the disclosed technology, the system further incudes a buckle configured to connect the at least one handle to the at least one band; and a restraint. The restraint incudes a first sleeve configured to receive overlapping portions of the at least one band; a second sleeve configured to receive a portion of a strap of the at least one handle; and a tether connected to the first and second sleeve and configured to straddle the buckle.

In another aspect of the disclosed technology, the restraint is restricted from substantial relative movement in lengthwise directions of the strap and the at least one band by interference between the first sleeve and the buckle, interference between the second sleeve and the buckle, and mutual restraint of the first and second sleeves by way of the tether.

In another aspect of the disclosed technology, the system further includes a sleeve configured to receive overlapping portions of the at least one band, wherein the sleeve is fixed to only one of the overlapping portions of the at least one band.

In another aspect of the disclosed technology, the at least one band is a first band; and the system further includes a second band.

In another aspect of the disclosed technology, respective end portions the first and second bands overlap, are secured to each other, and define a loop; and the first and second bands are configured to be connected to the first arm of the beam by way of the loop.

In another aspect of the disclosed technology, the second computing device is further configured to display, on a real-time or near real-time basis, the force acting on the force sensor.

In another aspect of the disclosed technology, the second computing device is further configured to calculate and display a percentage of the exercise session that has been completed by the user.

In another aspect of the disclosed technology, the second computing device is further configured to calculate target values for a force to be applied to the at least one handle by the user, based on the performance of the user during the exercise session or during a previous exercise session.

In another aspect of the disclosed technology, the second computing device is further configured to calculate and display target values for a rate and a number of repetitive applications of a force to be applied to the at least one handle by the user, and to display an actual rate and an actual number of repetitive applications of the force applied by the user to the at least one handle.

In another aspect of the disclosed technology, the second computing device is further configured to recommend to a user a difficulty level of an exercise session based on performance of the user during one or more prior exercise sessions.

In another aspect of the disclosed technology, the second computing device is a smartphone.

In another aspect of the disclosed technology, an exercise system includes a force sensor having a load cell. The load cell has a beam configured to be coupled to at least one band, and to an anchoring point; and at least one strain gauge mounted on the beam. The force sensor also includes a first computing device communicatively coupled to the strain gauge and having a processor configured to determine a force acting on the force sensor based on an output of the strain gauge. The system further includes a second computing device communicatively coupled the first computing device and configured to display information relating to an exercise session performed on the system by a user.

In another aspect of the disclosed technology, a force sensor includes a load cell having a substantially S-shaped beam with a first and a second arm; and at least one strain gauge mounted on the beam. The load cell also incudes a first computing device communicatively coupled to the strain gauge and having a processor configured to determine a force acting on the force sensor based on an output of the strain gauge. The force sensor also includes a first strap having a first loop formed therein and configured to be coupled to a band. The first strap is connected to the first arm by way of the first loop. The force senor also includes a second strap having a second loop formed therein and configured to be coupled to an anchoring point. The second strap is connected to the second arm by way of the second loop.

### DESCRIPTION OF THE DRAWINGS

The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

FIG. 1 is a perspective view of a resistance device of an exercise system with the capability to tailor exercise programs to the fitness level of the user.

FIG. 2 is a diagrammatic view of the exercise system with the capability to tailor exercise programs to the fitness level of the user.

FIG. 3 is a diagrammatic view of a server of the exercise system shown in FIG. 2.

FIG. 4 is a flow diagram of depicting operation of the system shown in FIG. 2.

FIG. 5 is table including a non-exhaustive list of parameters that can be monitored and/or calculated by the system shown in FIG. 2, to help assess and track the user's performance level.

FIG. 6 is a perspective view of a suspension training device of an alternative embodiment of the system shown in FIG. 2.

FIG. 7 is a diagrammatic view of the alternative embodiment of the system shown in FIG. 2.

FIG. 8A is a perspective, partial exploded view of a force sensor of the suspension training device shown in FIGS. 6 and 7.

FIG. 8B is a front view of the force sensor shown in FIG. 8A, with a front cover of the force sensor removed.

FIG. 9A is a magnified front view of the area designated “A” in FIG. 6.

FIG. 9B is a perspective view of the area designated “A” in FIG. 6.

FIG. 10A is a perspective view of end portions of a band of the suspension training device shown in FIGS. 6-9B, where the band is equipped with a sleeve for discouraging separation of the end portions.

FIG. 10B is a perspective view of the end portions and the sleeve shown in FIG. 10A, depicting one of the end portions at the maximum extent of its travel in relation to the other end portion.

FIG. 11 is a diagrammatic illustration of various electrical and electronic components of the force sensor shown in FIGS. 8A and 8B.

FIG. 12 is a top view of an anchor of the suspension training device shown in FIGS. 6-11.

FIG. 13 is a perspective view of the suspension training device shown in FIGS. 6-12, anchored to an anchoring point, depicting a user exerting force on the suspension training device.

FIG. 14 is a diagrammatic depiction of the deflection of a beam of the force sensor shown in FIGS. 8A and 8B, when the beam is subjected to an external force.

FIGS. 15 and 16 are perspective views of an alternative embodiment of bands of the suspension training device shown in FIGS. 6-14.

FIG. 17 is a diagrammatic depiction of the conversion of raw data into workable metrics by the systems shown in FIGS. 2 and 7.

FIG. 18 is a plot of percent body weight vs. time for an active exercise performed on a repetitive basis, with a trend line imposed on the data.

FIG. 19 is a plot of percent body weight vs. time for a static exercise, with a trend line imposed on the data.

FIG. 20 is a plot of percent body weight vs. time for a static exercise, with the data exhibiting relatively low residual values.

FIG. 21 is a plot of percent body weight vs. time for a static exercise, with the data exhibiting relatively high residual values.

FIG. 22 is a plot of percent body weight vs. time for a chest press exercise performed on a repetitive basis, with the data exhibiting relatively high theoretical effort value.

FIG. 23 is a plot of percent body weight vs. time for a chest press exercise performed on a repetitive basis, with the data exhibiting relatively low theoretical effort value.

FIG. 24 is a table depicting various exercises that can be performed using the systems shown in FIGS. 2 and 7.

FIG. 25 is a table depicting various metrics that the systems shown in FIGS. 2 and 7 can be configured to calculate, display, archive, and use to help tailor exercise routines to the user's fitness level.

### DETAILED DESCRIPTION

The following discussion omits or only briefly describes conventional features of the disclosed technology that are apparent to those skilled in the art. It is noted that various embodiments are described in detail with reference to the drawings, in which like reference numerals represent like parts and assemblies throughout the several views. Reference to various embodiments does not limit the scope of the claims appended hereto. Additionally, any examples set forth in this specification are intended to be non-limiting and merely set forth some of the many possible embodiments for the appended claims. Further, particular features described herein can be used in combination with other described features in each of the various possible combinations and permutations.

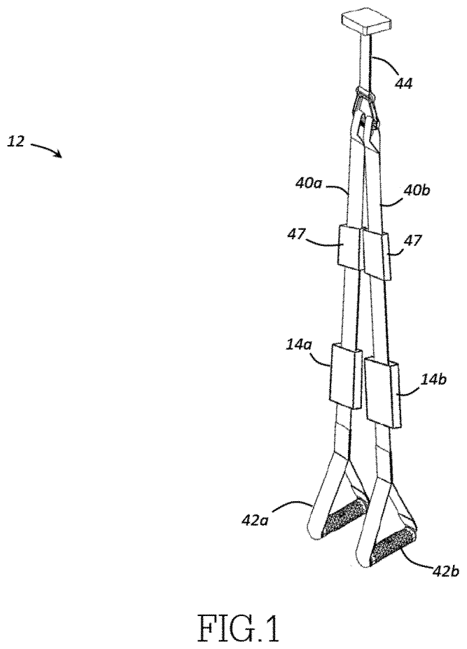

An exercise system 10 with the capability to tailor exercise programs to the fitness level of the user is disclosed. Referring to FIG. 1, the exercise system 10 comprises a resistance device 12, a first force sensor 14a, and a second force sensor 14b.

The resistance device 12 can include a first band 40a and a second band 40b. The resistance device 12 also includes a first grip 42a and a second grip 42b. The first and second bands 40a, 40b are formed from an elastomeric material, such as natural latex, that resiliently deforms when stretched. Each resistance band can generate a reactive force of, for example, about 13 lb. to about 22 lb. when stretched fully. The first force sensor 14a is connected to a first end of the first band 40a, and to the first grip 42a. The second force sensor 14b is connected to a first end of the second band 40b, and to the second grip 42b. A second end of each of the first and second bands 40a, 40b is connected to an anchor 44. The anchor 44 is configured to be connected to a stationary structure such as a wall. The anchor is formed from a material, such as woven nylon, that does not stretch substantially when subjected to a tensile force.

A user can generate a resistive force by pulling the first and second grips 42a, 42b away from the stationary structure to which the anchor 44 is attached, so as to stretch the first and second bands 40a, 40b. The resilience of the first and second bands 40a, 40b generates forces that resist the movement of the first and second grips 42a, 42b away from the anchor 44. These resistive forces increase as the first and second bands 40a, 40b are stretched further, causing the user to exert additional forces against the first and second grips 40a, 40b and thereby activating the muscle groups producing the movement of the first and second grips 42a, 42b. The resistance device 12 can include adjustment features 47 that permit the lengths of the first and second bands 40a, 40b between the anchor 46 and the respective first and second grips 42a, 42b to be adjusted, to allow the user to tailor the resistive forces generated by the first and second bands 42a, 42b to a particular exercise and body position of the user.

The first and second force sensors 14a, 14b measure and transmit the resistive forces generated by the first and second bands 42a, 42b to the respective first and second grip 42a, 42b. The first and second force sensors 14a, 14b each include a load cell that generates an electrical output related to the force being transmitted through the first or second force sensor 14a, 14b. Each of the first and second force sensors 14a, 14b also includes an electronics module communicatively coupled to the corresponding load cell. The electronics module provides an excitation voltage to the load cell. Also, the electronics module is configured to process the output of the load cell; and to generate force readings based on the response of the load cell to the applied loads and calibration data stored in the electronics module. The force readings for the first and second force sensors 14a, 14b correspond to the loads being applied by the user to the respective first and second grips 42a, 42b. The first and second force sensors 14a, 14b continually transmit the force readings to a user interface of the system 10 in the form of a smartphone 16, along with a unique identifier associated with the first or second force sensor 14a, 14b.

Specific details of the resistance device 12 are presented for illustrative purposes only. The system 10 can incorporate other types of resistance devices, and exercise devices other than resistance-training devices. Also, alternative embodiments of the resistance device 12 can include a single force sensor positioned between the anchor 46, and the first and second bands 40, 40b. Also, specific details of the first and second force sensors 14a, 14b likewise are presented for illustrative purposes only. The system 10 can incorporate other means for measuring the force exerted by the user on the first and second grips 42a, 42b, such as accelerometers.

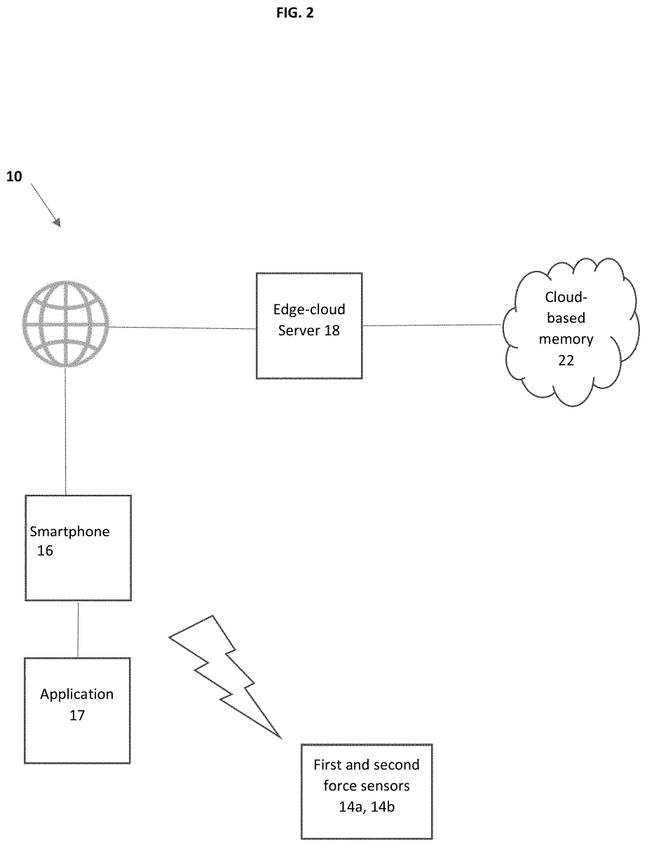

Referring to FIG. 2, the system 10 also includes a first computing device that can be accessed by the user during exercise programs. The first computing device can be, for example, the smartphone 16. Other types of computing devices, such as a tablet, a notebook, or a personal computer, can be used in lieu of the smartphone 16 in alternative embodiments.

The smartphone 16 is communicatively coupled to the first and second force sensors 14a, 14b by a suitable wireless means such as BLUETOOTH. The smartphone 16 includes an application or app 17 stored on a memory device of the smartphone 16. The app 17, when executed by a processor of the smartphone 16, facilitates communication between the smartphone 16 and the first and second force sensors 14a, 14b, and permits the smartphone 16 to act as a user interface for the system 10. The app 17, upon execution by the processor, also causes the smartphone 16 to perform the additional operations discussed below.

The system 10 further comprises a second computing device. The second computing device can be, for example, a server 18. Other types of computing devices, such as a mainframe computer, can be used in lieu of the server 18 in alternative embodiments. The server 18 can be positioned at a location remote from the resistance device 12 and the smartphone 16; and can be communicatively coupled to the smartphone 16 by a suitable communications network such as, but not limited to, the internet. The server 18 can be communicatively coupled to and can process data from multiple resistance devices 12 and multiple smartphones 16.

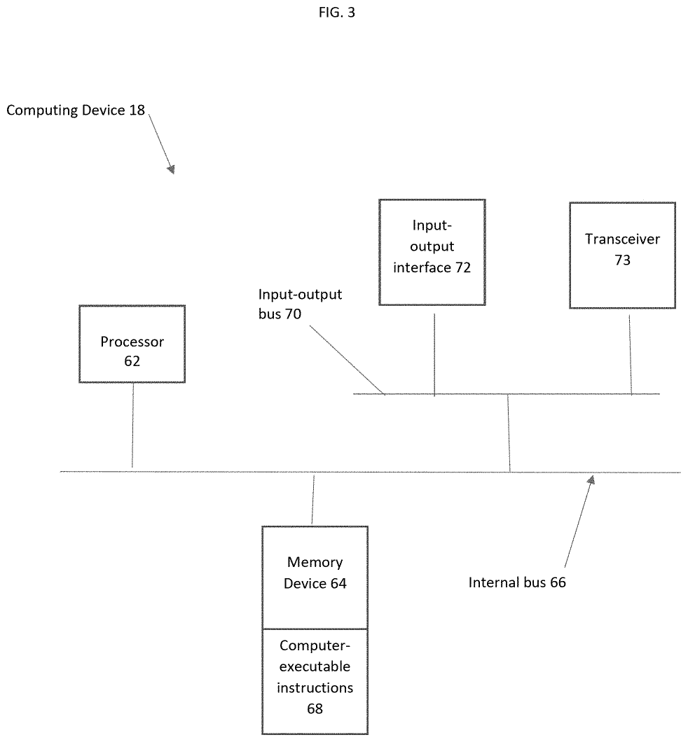

Referring to FIG. 3, the server 18 comprises a processor 62, such as a microprocessor; a memory device 64 communicatively coupled to the processor 62 via an internal bus 66; and computer-executable instructions 68 stored on the memory device 64 and executable by the processor 62. The server 18 also comprises an input-output bus 70; an input-output interface 72 communicatively coupled to the processor 62 by way of the input-output bus 70, and a transceiver 73 communicatively to the input-output interface 72. The computer-executable instructions 68 are configured so that the computer-executable instructions 68, when executed by the processor 62, cause the server 18 to carry out the various operations described herein. The above details of the server 18 are presented for illustrative purposes only. The server 18 has components in addition to those described above and can have an internal architecture other than that described above.

The server 18 can be communicatively coupled to a suitable cloud-based memory 22 of the system 10, shown in FIG. 2. The cloud-based memory 22 can be used, for example, to store archived data relating to the exercise history of the user. The cloud-based memory 22 also can be used to store various exercise programs indexed, for example, by the level of difficulty; the targeted muscle or muscle group; the user's fitness objective, etc. In alternative embodiments, the exercise programs and other information described herein as being stored on the cloud-based memory 22 can be stored on the memory device 64 of the server 18, on the memory device of the smartphone 16, or on another memory device.

The division of functions between the server 18 and the smartphone 16 as described below is presented for illustrative purposes only and is not intended to be limiting. Various functions described as being performed by the smartphone 16 can be performed by the server 18 in alternative embodiments. Likewise, various functions described as being performed by the server 18 can be performed by the smartphone 16 in other alternative embodiments. In other alternative embodiments, the functions of the smartphone 16 and the server 18 can be performed by one computing device.

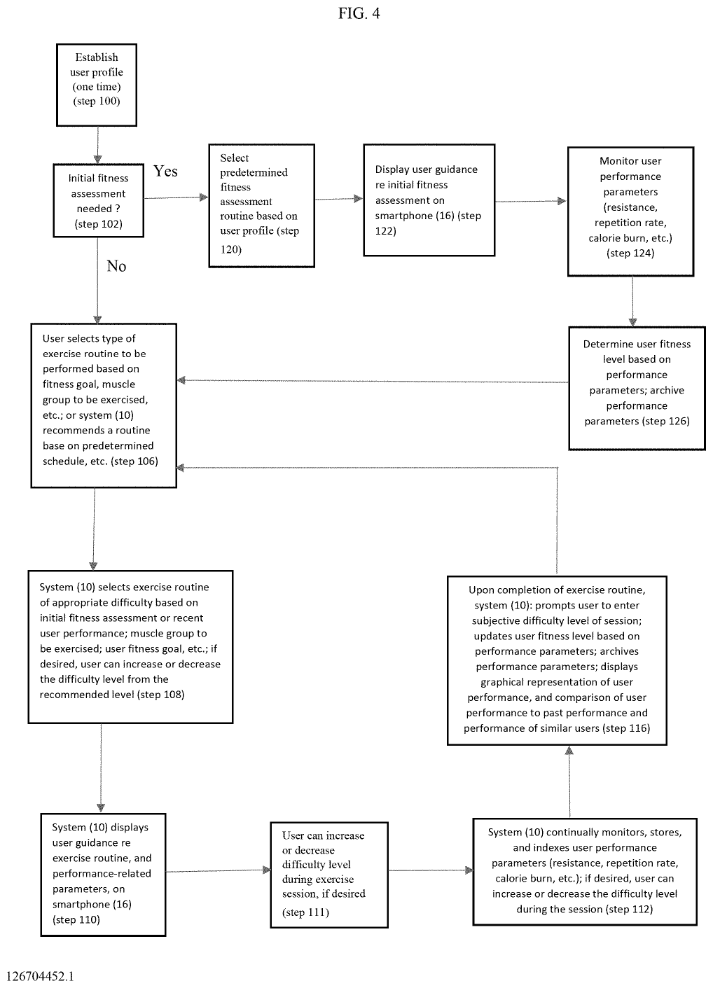

During use of the exercise system 10, the resistance readings provided by the first and second force sensors 14a, 14b are sampled continuously by the smartphone 16. The smartphone 16, executing the application 17, is configured to display the resistance readings on a real-time basis, so that the user can obtain instantaneous feedback regarding the level of force the user is exerting on the resistance device 12 (step 110 of the flow diagram depicted in FIG. 4). The smartphone 16 is further configured to display visual images and prompts that guide the user through a particular exercise program selected by the user. The smartphone 16 also can be configured to emit audible dialog and prompts to help guide the user through the exercise session. For example, the smartphone 17 can be configured to generate a voice cue notifying the user that the user has competed half of an exercise set, and another voice cue when the user has competed 90 percent of the exercise set.

The smartphone 16 continuously transmits the acquired force readings and the sensor identifier to the server 18. The smartphone 16 also transmits the identity of the user, and a timestamp associated with each force reading. The server 18 stores and indexes this information in the cloud-based memory 22, thereby creating a permanent archive of the exercise programs performed by the user, and the user's performance during each program (step 112).

The identify, age, height, weight, gender, and other relevant information about the user can be input using the smartphone 16, and can be stored on the smartphone 16, the server 18, and/or the cloud-based memory 22 as part of a user profile unique to each user. The user profile typically is established by the user prior to the user's first use of the system 10 (step 100 of FIG. 4).

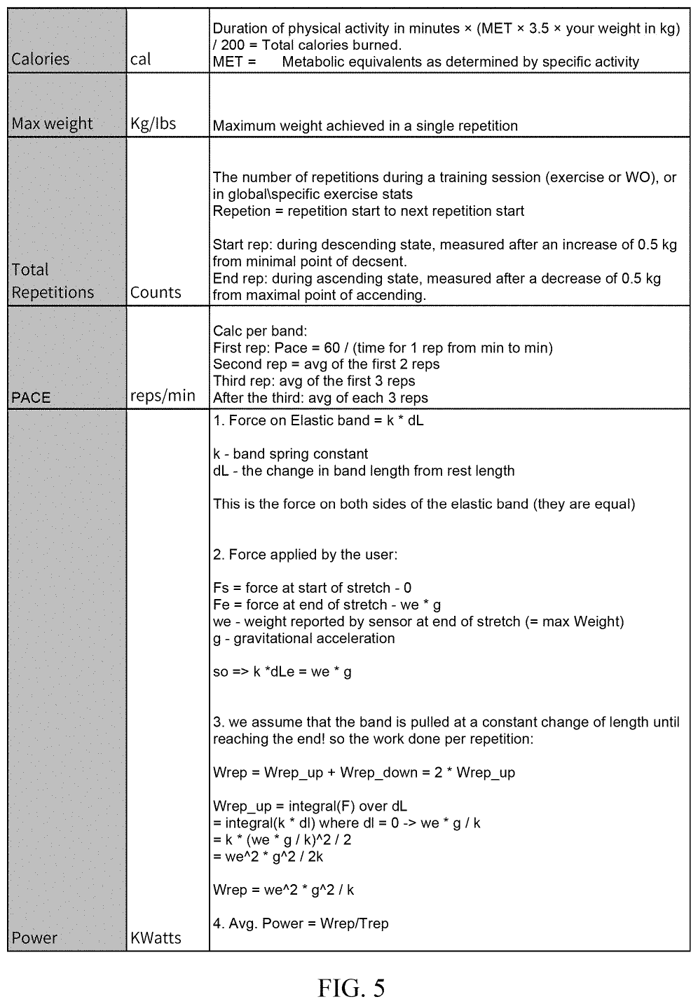

The data from each exercise session performed by the user can be stored in the memory 22, and can be indexed, for example, by the identity of the user, the targeted muscle or muscle group, the date the program was performed, etc. (step 112). The data can include the time-stamped resistance readings from the exercise session as obtained by the first and second force sensors 14. The data also can include other performance-related parameters measured or calculated by the system 10, such as the repetition rate of the exercises; total calories expended by the user; the overall duration, i.e., elapsed time, of the session; the user's heart rate; the work expended by the user; the power generated by the user; etc. Data is added each time user performs an exercise session, so that a permanent archive of that user's exercise history and performance is developed. FIG. 5 is a non-exhaustive list of various parameters that can be monitored and/or calculated by the system 10 to help assess and track the user's performance level.

The system 10 is configured to use the resistance readings generated by the first and second force sensors 14a, 14b, and other performance-related data, to adjust the level of difficulty of the upcoming exercise session so as to tailor user's exercise experience to the user's ability, i.e., to the user's fitness level (steps 108, 114). Also, the system 10 is configured to guide the user through the exercise program selected by the user (step 110). Based on the user's workout history and past performance, and the age, height, weight, gender, and/or other relevant characteristics of the user, the server 18 can recommend specific exercise programs for the user (step 106).

### Fitness Assessment

The system 10 is configured to guide the user through an optional fitness assessment, to help determine an appropriate level of difficulty in the exercise sessions to be performed initially by the user (step 102). Typically, the fitness assessment is performed by new users, i.e., by users without an exercise history archived by the system 10. Once a user has established a workout history using the system 10, the archived user data is evaluated each time the user commences an exercise session on the system 10, to assess the user's fitness level and recommend a particular exercise session based on the user's fitness level (step 108 of FIG. 4).

The smartphone 16, executing the app 17, can guide the user through the initial fitness assessment (step 122). The fitness assessment can be tailored, for example, to the age, gender, height, and/or weight of the user (step 120). The user, after establishing a user identification and entering the above personal information to establish a user profile, can initiate the fitness assessment via user-driven menus displayed on the smartphone 16.

Upon initiation of the fitness assessment, the smartphone 16, in conjunction with the server 18, chooses a predetermined fitness assessment session based on the user profile, i.e., based on factors such as the age, gender, height, and weight of the user (step 120 of FIG. 4). The fitness assessment session can be chosen from a database residing on the cloud-based memory 22 and accessed by way of the server 18. The lookup table incudes fitness assessment sessions indexed by the user's age, gender, height, weight, etc. Once the appropriate fitness assessment session is chosen, it can be uploaded to the smartphone 16. The smartphone 16 can display video and audio prompts to guide the user through the fitness assessment session (step 122).

For example, the user can be prompted to repeat a particular movement, with a particular weight or resistance, as quickly as possible over a predetermined time period such as one minute. The smartphone 16, executing the app 17, can monitor and interpret the force profile to determine the beginning of each repetitive movement, and can monitor the time stamps of the force readings to calculate the rate at which the user is performing the repetitions (step 124). The server 18 can assess the user's fitness level based on, for example, the time between repetitions. A separate assessment process can be performed for different muscle groups. For example, fitness assessments can be performed for the user's upper body, lower body, and core.

Alternatively, the user can be prompted to repeat a particular movement, with a particular weight or resistance, at a constant pace set by the system 10, until the system 10 determines that the time between repetitions increases by a predetermined amount, e.g., by about 50 percent. The server 18 can assess the user's fitness level based on, for example, the elapsed time or the number of repetitions performed before the time between repetitions has increased by the predetermined amount.

Upon completion of the fitness assessment, the server 18, executing the computer-executable instructions 68, can compare the user's performance with the average performance of other users with similar characteristics performing the same or a similar fitness assessment session (step 126). For example, the user's performance can be compared with the performance of other users of the same gender, and of similar height, weight, and/or age. Upon determining the user's relative fitness level, the server 18 can generate recommendations for specific exercise programs (step 108). More particularly, the server 18 can match the fitness level, age, height, and/or weight of the user with appropriate exercise programs based on the indexed exercise sessions stored in the cloud-based memory 22. These recommendations can be provided to the user by way of the smartphone 16.

### User-Tailored Exercise Program

The application 17 of the smartphone 16 can be configured to cause the smartphone 16 to display a series of interactive menus that can guide the user through the various features the system 10. For example, one menu sequence can permit the user to select a type of exercise program tailored a particular fitness goal of the user, and a particular muscle group or muscle (step 106 of FIG. 4). Also, the system 10 automatically can guide the user to a particular exercise program based on, for example, an exercise schedule previously input by the user, a fitness session previously chosen by the user, etc. (step 106). Other menu sequences can guide the user to graphical depictions of the user's performance level during a past exercise program completed by the user; trends in the user's performance level; a listing of recently-completed exercise sessions along with the calories consumed during the programs; other archived data; etc.

The server 18, executing the computer-executable instructions 68, can recommend specific exercise programs for a particular user based on, for example, the results of the fitness assessment, the user's fitness goals, the targeted muscle or muscle group, the user's performance during recent exercise sessions, etc. (step 108). Specifically, the server 18 can access a database of exercise programs that are stored in the cloud-based memory 22. The programs can be indexed, for example, by the targeted muscle or muscle group; the recommended fitness level of the user; the fitness goal of the user; etc. The targeted muscle or muscle groups can include, for example, upper body, lower body, core, biceps, triceps, shoulders, legs, chest, glutes, legs, abs, back, etc. The fitness goal can include one or more of, for example, weight loss; getting fit; strength; flexibility and mobility; building muscle; improving health; maintaining fitness, burning fat, etc.

The exercise sessions can be, for example, live or pre-recorded sessions with an instructor, animations illustrating the particular exercise movement to be performed, etc. The system 10 can guide the user through, for example, the number of repetitions, the pace of the repetitions, the force exerted during each repetition, the elapsed time of the exercise session, etc. For example, system 10 may instruct the user to perform one repetition every 20 seconds, for a predetermined period of time or a predetermined number of repetitions. The force can be selected, for example, by instructing the user to employ a particular type of first and second band 40a, 40b, and to select the length of the first and second bands 40a through the adjustment features 47.

Once a particular type of exercise session has been selected by the user or recommended by the system 10, the server 18, executing the computer-executable instructions 68, tailors the difficulty level of the exercise session to the user's fitness level (step 108). For new users without an established archive of data from previous workouts, the above-noted initial fitness assessment can be used as an indication of the user's fitness level.

For users with an established archive of data from previous workouts, the server 18, executing the computer-executable instructions 68, looks up the archived user data from the exercise sessions most recently completed by the user, and selects a session of appropriate difficulty based on the performance-related parameters measured during the most recent sessions completed by the user (step 108). For example, the server 18 can tailor the level of difficulty of the upcoming exercise session based on a score generated after the user's most recent exercise session or sessions. The score can be a composite index calculated based on one or more of the following performance-related parameters: the measured force or resistance exerted by the user; the repetition rate of the individual movements; the energy (calories) consumed by the user; the duration or elapsed time of the session; the users' average or maximum heart rate; etc.; the overall work performed by the user; the power exerted by the user, etc. If desired, the user can increase or decrease the difficulty level of the exercise session from the recommended level, by entering inputs via the smartphone 16.

The distance through which the user applies force to the first and second grips 42a, 42b during a particular movement is needed to calculate the work and power associated with the movement. The distance can be estimated using a lookup table in which the measured force is correlated with the distance or deflection of the first or second grip 42a, 42b needed to produce that force. The lookup table can include difference sets of force-deflection data corresponding to different initial lengths of the first and second bands 40a, 40b. For example, the system 10, via the display on the smartphone 16, can prompt the user to place the adjustment features 47 in a particular position, e.g., short, medium, or long, at the beginning an exercise session. For the purpose of calculating work and power, length of the first and second bands 40a, 40b can be assumed based on the position to which the user is prompted to move the adjustment features 47.

Based on, for example, the muscle group to be exercised, the user's fitness goal, and the fitness level of the user, the server 18 identifies a particular type of exercise session from a database residing on the cloud-based member 22; and based on the user's score during the most recent exercise session or sessions completed by the user, the server selects a specific exercise session with a predetermined difficulty rating appropriate for the user's score or scores (steps 106, 108). The exercise session is uploaded to the smartphone 16. The smartphone 16 can display video and audio prompts to guide the user through the fitness assessment session (step 110).

The smartphone 16, executing the app 17, is configured to monitor and process, on a real-time basis, the resistance readings generated by the force sensors 14 in response to the forces exerted by the user on the first and second grips 42a, 42b (step 112). For example, the smartphone 16 can generate a time-varying profile of the combined resistance readings as a repetitive exercise is being performed by the user. The smartphone 16 can recognize a smooth, sinusoidally-varying profile in the resistance as an indication that the user is not struggling during that portion of the exercise program. Conversely, deviations from a smooth, sinusoidally-varying profile are interpreted as an indication that the user is struggling to perform the exercise, and is approaching or has exceeded the limit of the user's performance. The smartphone 16 can generate a notification to the user upon detecting such a decline in the user's performance. The notification can be a visual notification displayed on the smartphone 16, and/or an audible indication generated by the smartphone 16.

The smartphone 16 is further configured to monitor other performance-related parameters, such as but not limited to the user's heat rate; total duration, i.e., elapsed time, of the exercise session; time between repetitions; other parameters listed in FIG. 5, etc. (step 112). Also, the smartphone 16 can calculate the calorie burn of the user, the work performed by the user, and the power generated by the user.

If desired, the user can increase or decrease the difficulty level of the exercise session during exercise session, by entering inputs via the smartphone 16 (step 111). In alternative embodiments, the smartphone 16, executing the app 17, can be configured to adjust, or modify the exercise session in real time based on the performance of the user, i.e., based on whether the user's performance is at, above, or below the expected level for the particular workout session being performed. In assessing the user's performance, the smartphone 16 can consider, without limitation, one or more of the following factors: the above-noted force-time profile of the measured resistance levels; the actual resistance level being exerted by the user, the repetition rate of the movements; the user's heart rate and calorie burn rate, etc.

Upon completion of the exercise session, the server 18, executing the computer-executable instructions 68 and accessing user data archived in the memory 22, can compare the user's performance to the prior performance of the user during recent, similar sessions, to assess any improvement or degradation in the user's fitness level (step 116). The server 18 updates the user's fitness level to reflect the data obtained during the most recent exercise session and can provide the user with recommendations for subsequent exercise sessions based on the updated fitness level. For example, if the user's performance during the most recent exercise session meets or exceeds the expected performance level, the server 18 can proportionally increase the difficulty level of subsequent exercises, i.e., the server 18 can set new targets that challenge the user and help the user stay on track to achieve the user's fitness goals.

Also, upon completion of the exercise session, the smartphone 16 can prompt the user for input regarding the difficulty of the exercise routine (step 116). For example, the user can be prompted to rate the difficulty of the exercise session on a numerical scale of one to ten. The server 18 can use this information in addition to the user's actual measured performance to assess the user's fitness level and tailor the subsequent exercise routines to the user's fitness level.

The user can access and review the performance data on the smartphone 16 immediately after completing the exercise session, or at a later time, using the menu-driven displays on the smartphone 16 (step 116). Also, the server 18 can generate a comparison or ranking the user's performance in relation to other users of similar age, gender, height, and/or weight, using the user data archived in the memory 22. The comparison or ranking can be displayed on the smartphone 16. The progress of the user and/or the ranking of the user can be displayed, for example, using graphics such as bar charts or two-axis plots.

### Display

The smartphone 16, executing the app 17, can display various parameters relating to the user's performance during the exercise program (step 110). For example, a real-time graphical representation of the resistance offered by the exercise device 12, as determined by the first and second force sensors 14a, 14b, can be displayed along with the video. The graphical representation can be, for example, a circular or curvilinear gauge with a curser that moves along the circumference or the length of the gauge to indicate the resistance level at any given time; or a triangle whose three legs extend proportionally to indicate the user's performance in relation to the user's upper body, lower body, and core muscle groups. Also, a graphical representation of the pace of the exercise session, as indicated by the number of repetitions per minute, can be displayed, for example, as a vertical bar that rises and falls with the number of repetitions per minute.

The smartphone 16, executing the app 17, also is configured to calculate and display a running total of the aggregate energy expended by the user, in calories, over the course of the exercise program. The calculation is based on the time-stamped resistance readings. Other parameters that can be tracked and displayed include running totals of the number of repetitions and sets performed during the program, the total elapsed time of the exercise program, the aggregated time spent applying force to the resistance device 12, the muscle groups being activated by a particular workout, the work and power performed or produced by the user, etc.

### User Progress

The server 18, executing the computer-executable instructions 68, can generate a score of the user's performance over the course of the exercise session. The score can be generated based on, for example, a composite index of various performance metrics such as, but not limited to the number of repetitions; the pace of repetitions; the average force exerted by the user; the work performed during the session; the power exerted by the user during the session; other parameters listed in FIG. 5, etc.

The system can compare the user's performance during a particular exercise program with the user's past performance (step 116). Specifically, upon completion of the exercise program, the server 18 can look up archived scores and other archived data corresponding to the same or similar type of exercise programs previously completed by the same user. The server 18 can compare the user score during the latest exercise session with the scores achieved during the previous programs. Also, the server 18 can compare various exercise parameters, such as the resistance readings and the frequency of the repetitions, with the corresponding parameters as measured during the previous programs. The server 18 can recognize trends indicating increases or decreases in the user's performance. For example, the server 18 can recognize a predetermined increase in the user's overall score as an indication that the user's performance has increased with respect to the muscle or muscle group targeted by that particular exercise session. The user's score and other performance-related information can give the user an indication of his or her fitness level, and the progress of the user toward his or her fitness goals. Also, as discussed below, the user score can be used by the system 10 in selecting an exercise session of appropriate difficulty during the user's next exercise session.

The system 10 can be configured to rank the user in comparison to other users of the same gender, and of similar height, weight, and age, based on the performance-related information generated during the user's exercise session. The system 10 can generate weekly challenges, and can encourage competition among users by, for example, posting user scores on a leaderboard after obtaining permission from the users to do so. Thus, the system 10 facilitates personalization of the user's fitness program based on user performance, user feedback, and inputs from other users.

Based on a favorable, i.e., increasing, user score for a particular exercise session in comparison to prior scores for similar sessions, the server 18 can tailor a recommended exercise session for the relevant muscle or muscle group to present the user with a more challenging exercise session suitable for the user's enhanced performance level, to further advance the user's performance level during subsequent exercise sessions and help maximize the fitness gains of the user. Conversely, if the server 18 detects a decrease in the user's score, the server 18 can tailor the recommend exercise program to present a less challenging exercise session, to help minimize the possibility for injury. The server 18 automatically can recommend an exercise session of an appropriate level of difficulty after the user enters the muscle or muscle group to be exercised, and the user's fitness objective during the exercise session being initiated by the user. For example, the recommended resistance and/or the repetition rate of the movements in the exercise session can be increased or decreased to vary the difficulty of the session.

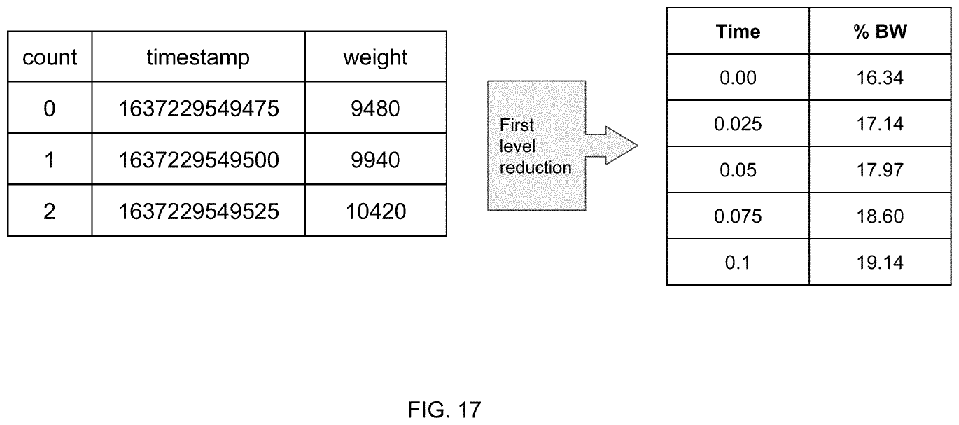

The server 18 and/or the smartphone 16 can be configured to convert raw data into workable metrics, as follows. Raw data are read as: a count; a timestamp in milliseconds; and a weight, or measured force, in grams, as depicted in FIG. 17. A first level reduction then is performed by removing the count (which is used only for event identification); converting the timestamps into a running time, in seconds; and converting the weight to a percentage of the body weight of the user, or “percent body weight” (which is of particular importance for analysis of date generated by suspension training).

Data can be gathered and analyzed in connection active exercises and static exercises. Active exercises are exercises in which the user moves one of more body parts, usually in a repetitive manner, and varies the force exerted by the user on the handles or other input device. Static exercises are exercises in which the user does not move, or attempts to not move, while maintaining, or attempting to maintain a substantially constant force on the handle or other input device.

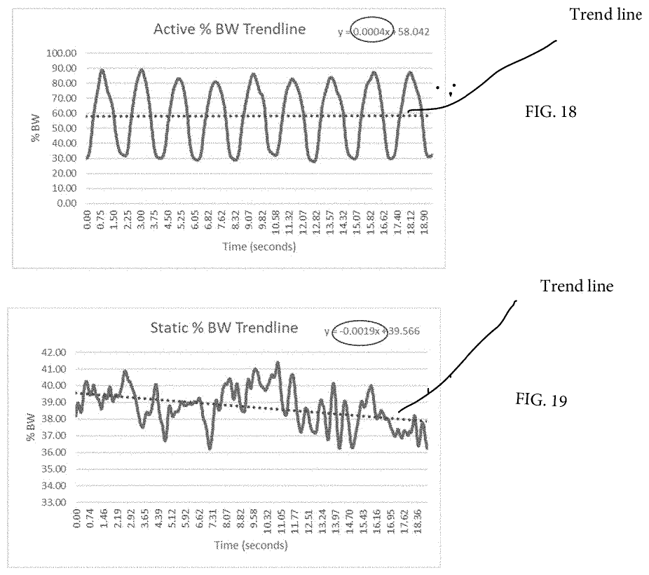

FIG. 18 is a plot of the percent body weight vs. time for an active exercise performed on a repetitive basis. As can be seen in this figure, the data forms a clearly discernable waveform; and the repetitions are clear and measurable, based on established threshold criteria.

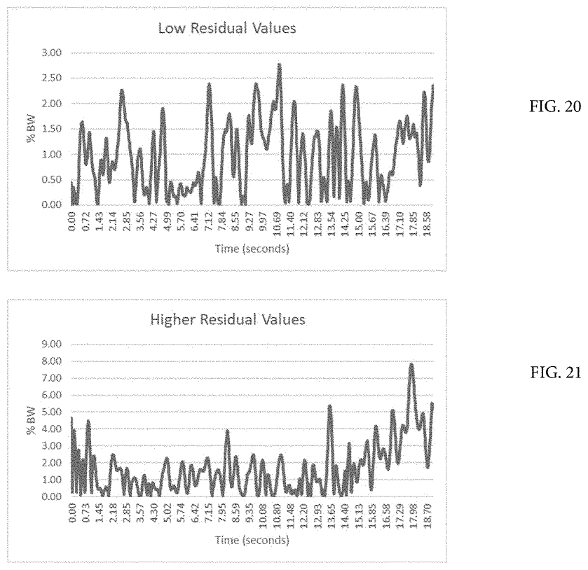

FIG. 19 is a plot of percent body weight vs. time for a static exercise performed on a repetitive basis. As can be seen in this figure, repetitions are not discernible, and the data is not amenable to setting threshold values and cannot be measured.

The server 18 and/or the smartphone 16 also can be configured to generate trend lines from the data. For example, FIG. 18 depicts a trend line overlayed on the active percent body weight vs. time data. FIG. 19 depicts a trend line overlayed on the static percent body weight vs. time data. The trendline is a way of measuring percent body weight over time. The trend lines are based on the following linear regression: y=mx+b, where b represents the intercept, i.e., the value of percent body weight at which the trendline intersects the y axis at the beginning of data recording. The intercept y represents the midpoint of the force curve, as measured by percent body weight, at the beginning of the movement.

The parameter m represents the slope, and indicates how the midpoint of the force curve, as measured by percent body weight, changes from the beginning to the end of the exercise. The intercept b and the slope m both can be used to establish baseline references for all exercise types. The intercept b can establish a target percent body weight against which to measure performance, and the slope can determine whether the user's effort is optimal, with a slightly negative slope indicating optimal exercise effort.

The server 18 and/or the smartphone 16 also can be configured to perform a residual analysis. A residual is the difference between an actual value, and the predicted trendline value for that moment in time. Residuals denote variability around the trendline. The bigger the residual values, the more the force is varying. As residuals get closer to the trendline, the activity gets consistently more stable.

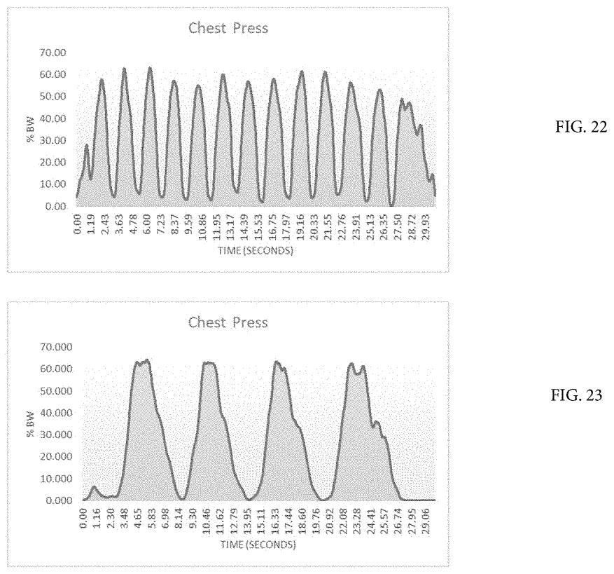

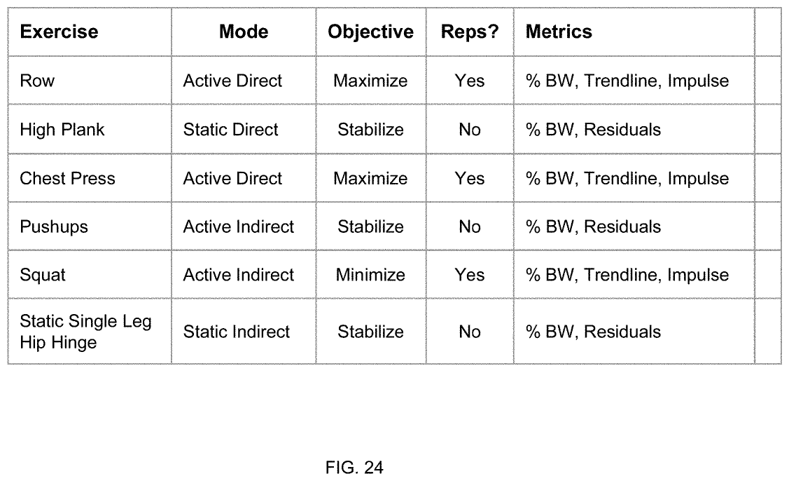

In static exercises, a primary objective is to eliminate force variability by keeping the handles as still as possible. Thus, a baseline force value for a particular static exercise can be established, and user performance can be assessed by comparing the actual value against the baseline value. For example, in the data presented in FIG. 20, the average residual value is 0.96 percent body weight and the peak residual value is less than 3.0 percent body weight, indicating of a high level of static control. In the data presented in FIG. 21, by contrast, the average residual value is 1.67 percent body weight and the peak residual value is about 8.0 percent body weight, indicating of a lower level of static control.

The server 18 and/or the smartphone 16 can be configured to calculate impulse. Impulse is the total area under the curve of the force waveform, and represents the total effort generated during the exercise. The impulse is the product of the magnitude of percent body weight and the time interval over which the force is generated. Essentially, impulse is force multiped by time, or kg-second. Baseline reference values for impulse can be established for all active exercises, and individual performance can be measured against the baseline reference values.

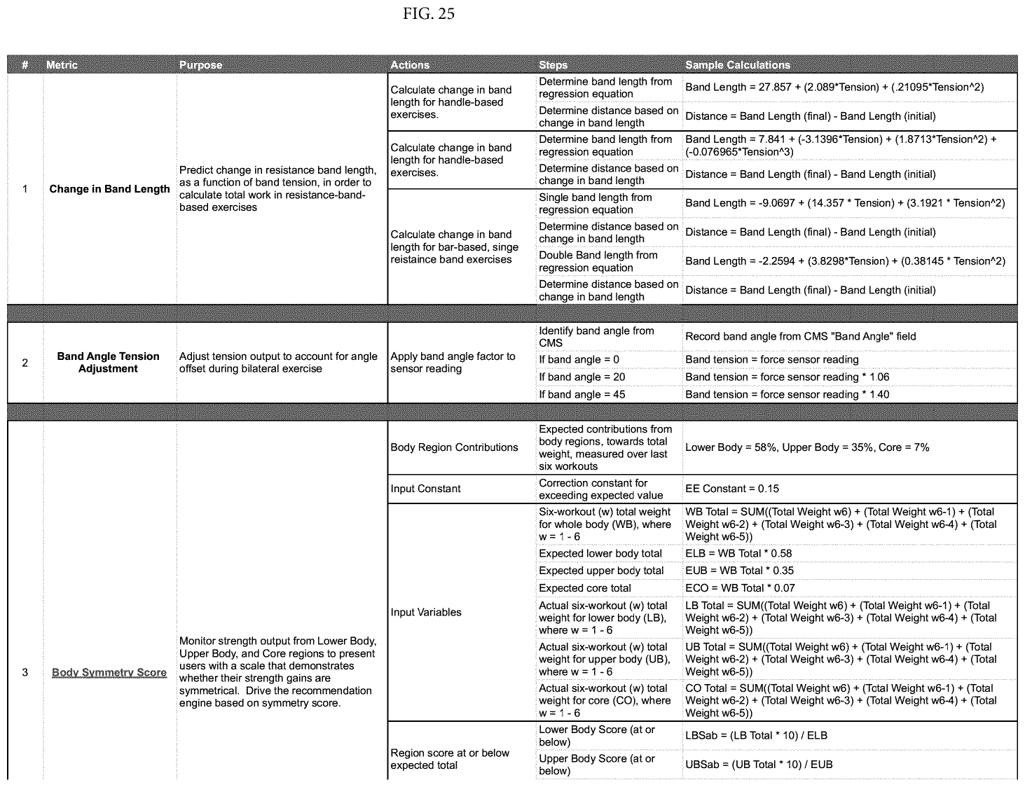

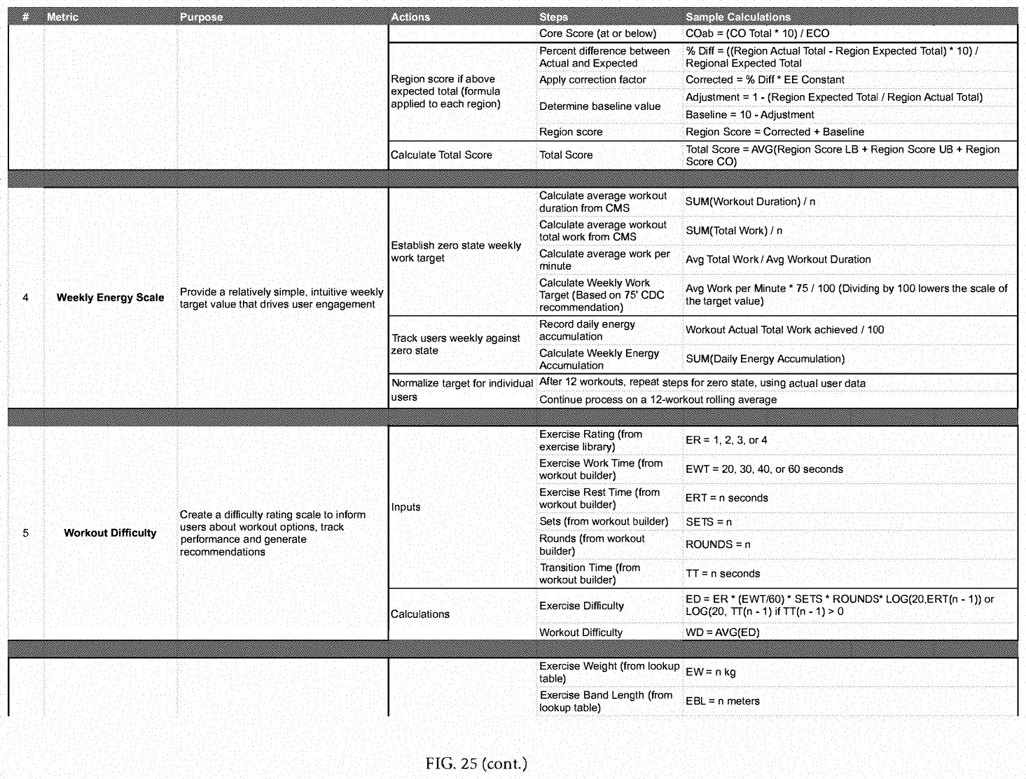

The server 18 and/or the smartphone 16 can be further configured to determine a theoretical “effort” value for active exercises. For example, in the data depicted in FIG. 22, the total impulse, or area under the curve, is 960.73 kg-s. The data indicates that the user completed 13 repetitions over the prescribed time, at a fairly consistent force output, and had a relatively high total effort. In the data depicted in FIG. 23, by contrast, the total impulse is 743.82 kg-s. Although the user maintained the same maximum force output as reflected in the data of FIG. 22, the user was able to complete only four repetitions in the prescribed time, and thus had a lower total effort in comparison to the exercise routine reflected in the data of FIG. 23.

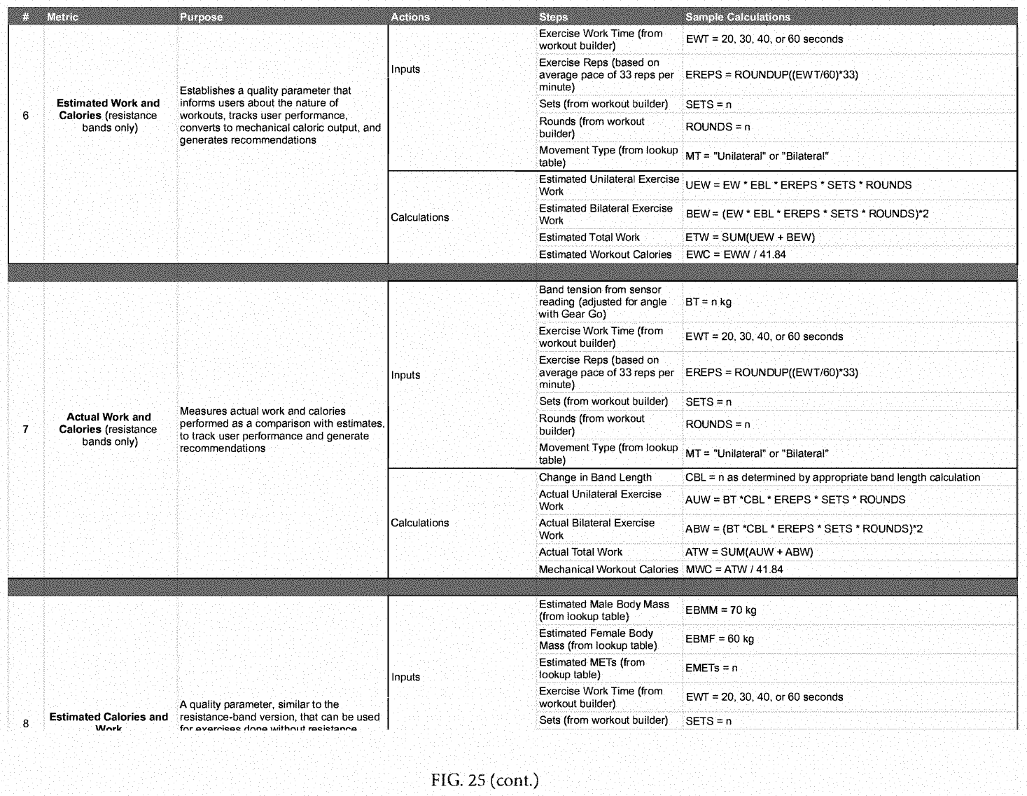

FIG. 24 is a table depicting various exercises that can be performed using the system 10, along with characteristics of the exercises and metrics associated with the exercises.

The above-noted parameters can be displayed on the smartphone 16 on real-time or near real-time basis; can be recorded and archived; and can be used by the smartphone 16 and/or the server 18 to track the user's progress and fitness level, and to help tailor exercise routines to the user's fitness level.

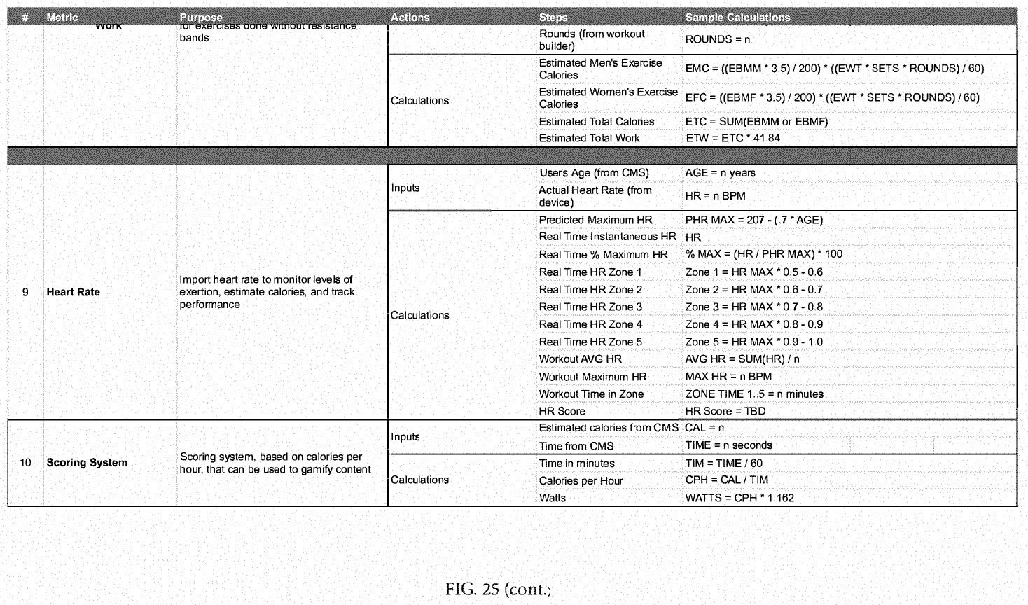

FIG. 25 is a table depicting various additional metrics that the server 18 and/or the smartphone 16 can be configured to calculate, display, archive, and use to help tailor exercise routines to the user's fitness level.

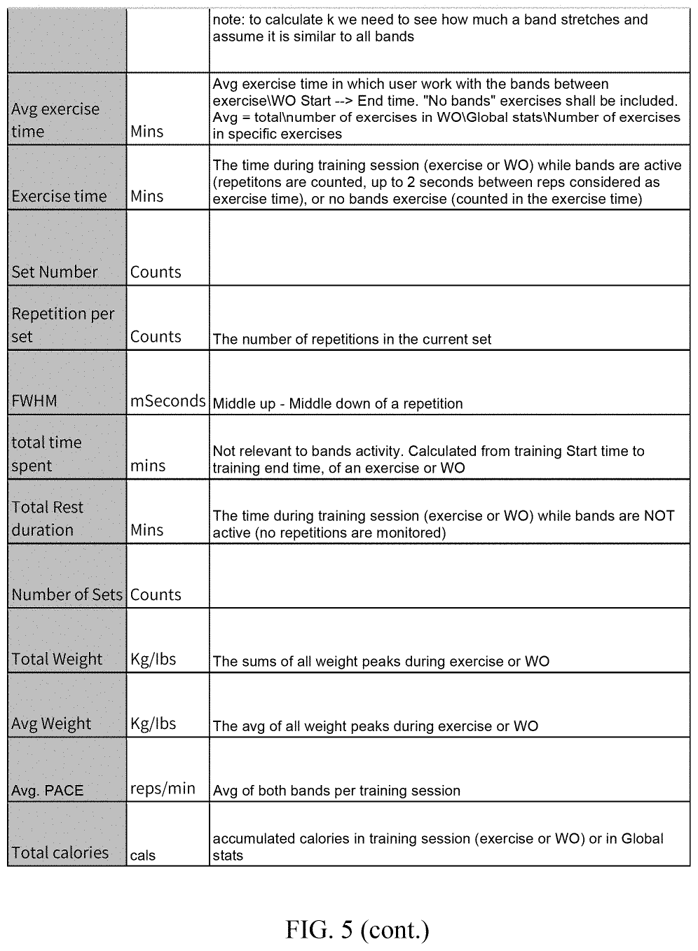

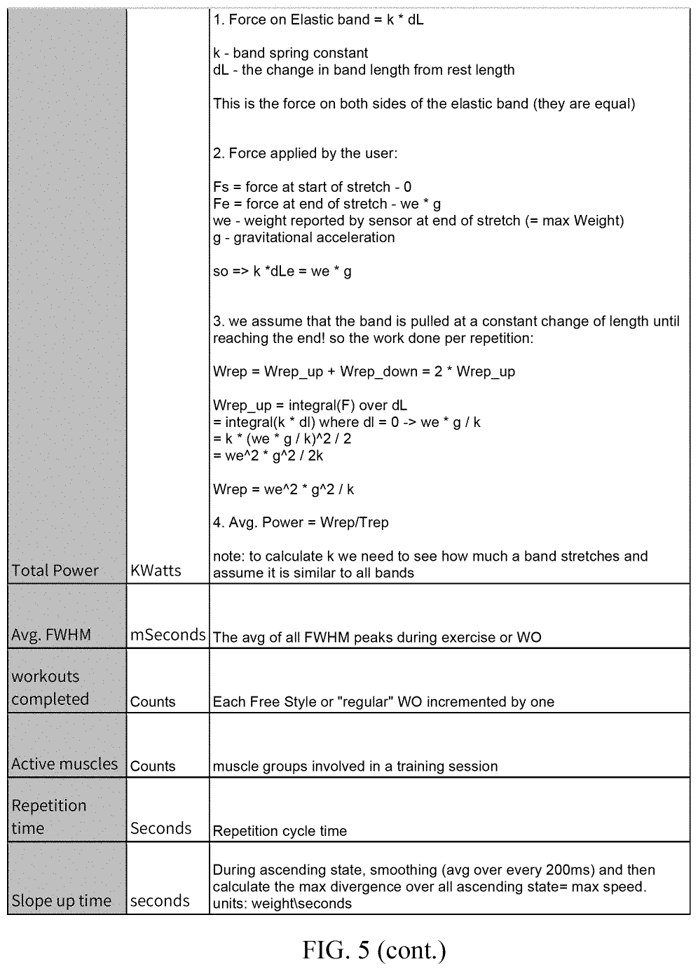

FIGS. 6-14 depict an alternative embodiment of the system 10 in the form of a system 200. The system 200 includes the smartphone 16, sever 18, and cloud-based memory 22 as discussed above in relation to the system 10. Except where otherwise noted, above descriptions of the functional, structural, and other aspects of the system 10 apply equally to the system 200.

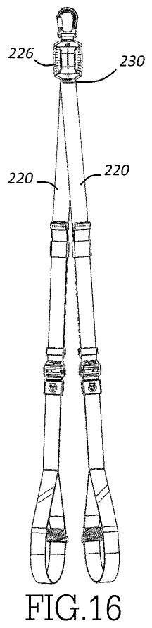

Referring to FIG. 6, the system 200 includes a resistance device in the form of a suspension training device 202. The suspension training device 202 comprises two bands 220, a force sensor 226, and two handles 228. The bands 220 are formed from an inelastic, flexible material such as nylon. A first end of each band 220 is folded back upon itself and stitched to the underlying portion of the band 220, to form a loop 230. As discussed below, the loops 230 facilitate attachment of the bands 220 to the force sensor 226. In alternative embodiments, the bands 220 can be equipped with a separate means for attaching the bands 220 to the force sensor 226. For example, a D-ring of a carabiner can be attached securely to the first end of each band 220. In other alternative embodiments, portions of the bands 220 proximate the respective first ends of the bands 220 can be made to overlap. The overlapping portions can be folded back upon themselves and stitched together to form a single loop 231, as shown in FIG. 15. The bands 220 can be connected directly to the force sensor 226 by way of the loop 230, as shown in FIG. 16.

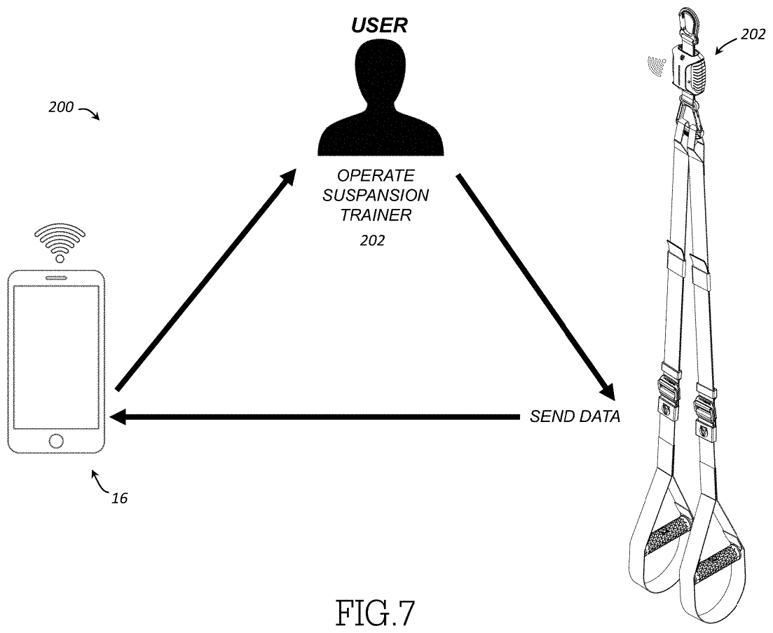

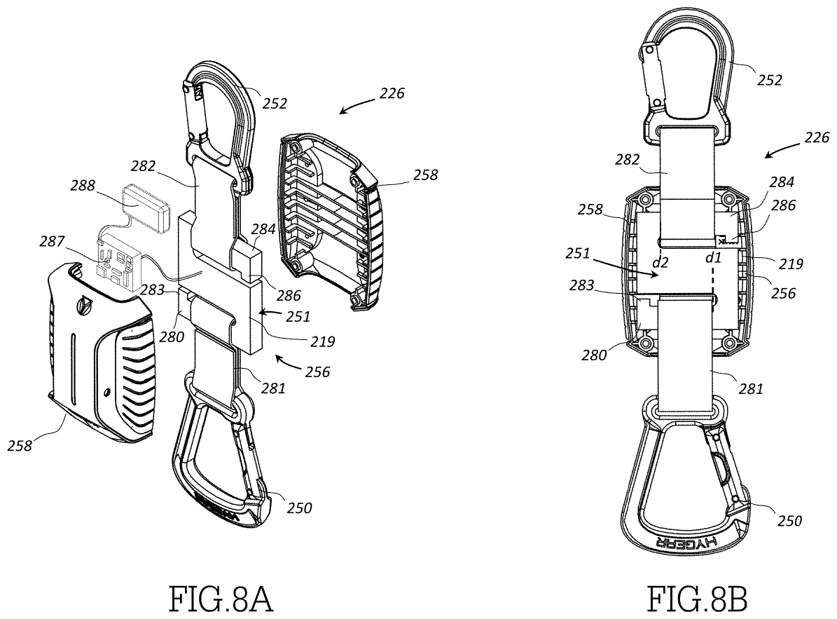

Each handle 228 is configured to act as both a hand grip and a foot cradle for the user; and the term “handle,” as used throughout the specification and claims, is intended to encompass hand grips and foot cradles. The handles 228 each include a strap 240 formed from an inelastic, flexible material such as nylon. As can be seen in FIG. 6, the strap 240 is folded back onto itself to form a relatively large loop 242; and a first end 239a of the strap 240 is secured to a portion of the strap 240 proximate a second end 239b of the strap 240 by a suitable means such as stitching. The second end 239b of the strap 240 is folded back onto itself to form a relatively small loop 241. The second end 239b and the loop 241 is visible in FIGS. 9A and 9B. The strap 240 is formed from an inelastic, flexible material such as nylon.

Referring again to FIG. 6, each handle 228 also includes a rigid hand grip 243 located within the loop 241. Each hand grip 243 is securely attached to the strap 240 by a second strap 245 that extends through the hand grip 242. Opposite ends of the strap 245 are attached to the strap 240 by a suitable means such as stitching, to form a secure connection between the hand grip 243 and the strap 240. The strap 245 is formed from an inelastic, flexible material such as nylon.

Details of the 228 handles are presented for illustrative purposes only. Alternative embodiments of the system 200 can include handles having other configurations, including handles in the form of a rigid bar.

Referring to FIGS. 9A and 9B, each handle 228 is secured to its corresponding band 220 by a two-piece buckle 290. In particular, the loop 241 of the handle 228 is connected to an arm 291 of a first portion 292 of the buckle 290, and an arm 293 of a second portion 294 of the buckle 290, so that the arms 291, 293 extend through, and engage the loop 241.

The band 220 extends through respective openings in the first and second portions 292, 294 of the buckle 290; and the band 220 is looped around an another arm 295 of the first portion 292.

Thus, force applied by the user to the handle 228 is transmitted to the buckle 290 by way of the loop 241 of the handle 228, and the arms 291, 293. The force is transmitted from the buckle 290 and to the band 220 by way of the second portion 294 of the buckle 249, and the arm 295 of the first portion 292.

The user can adjust the effective length of each band 220, i.e., the distance between the respective attachment points of the handle 228 and the force sensor 226 to the band 220, by varying the location on the band 220 at which the band 220 loops over and around the arm 295 of the first portion 292 of the buckle 290. The length adjustment can be performed when the band 220 is not under tension. When the band 220 is under tension, e.g., when the user-generated force is being transmitted to the force sensor 226 by way of the band 220, the portion of the band 220 located between the first portion 292 and the second portion 294 is pressed against, and restrained from relative movement by frictional contact with the adjacent surfaces of the first and second portions 292, 294, as can be seen in FIG. 9B.

The handles 228 can be coupled to their respective bands 220 by means other than the buckles 290 in alternative embodiments.

Referring still to FIGS. 9A and 9B, the suspension training device 202 further comprises a restraint 320. The restraint 320 is formed from an elastic material such as silicone. The restraint 320 causes the overlapping portions of the first band 220 proximate the buckle 290 to remain in contact. The restraint 320 comprises a first sleeve 322 that encloses the overlapping portions of the first band 220 proximate the buckle 290, and urges the overlapping portions into contact with each other.

The restraint 320 also comprises a second sleeve 324 that encloses the portion of the strap 240 proximate the loop 241. The restraint 320 further includes a tether 326. As can be seen in FIG. 9B, the tether 326 straddles the buckle 290, and connects the first and second sleeves 322, 324. The restraint 320 is restricted from substantial relative movement in the lengthwise directions of the strap 240 and the band 220 by interference between the first sleeve 322 and the buckle 290; interference between the second sleeve 324, and the loop 241 and the underlying portion of the buckle 290; and the mutual restraint of the first and second sleeves 322, 324 by way of the tether 326.

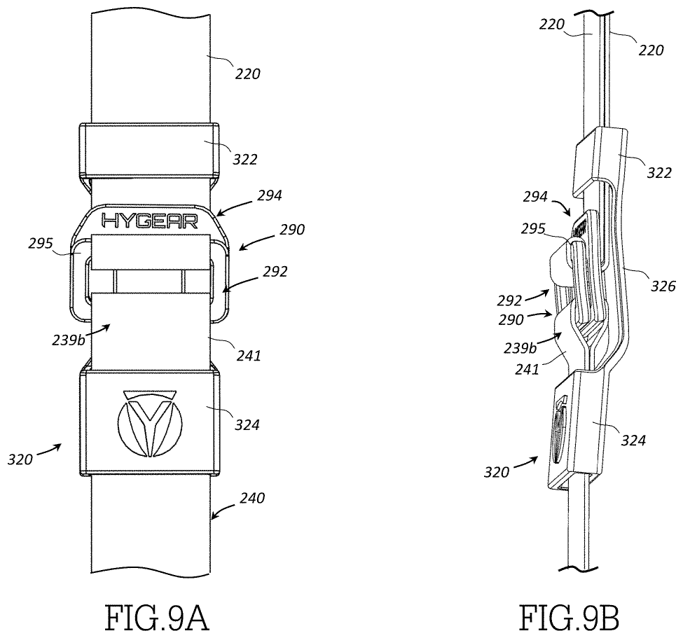

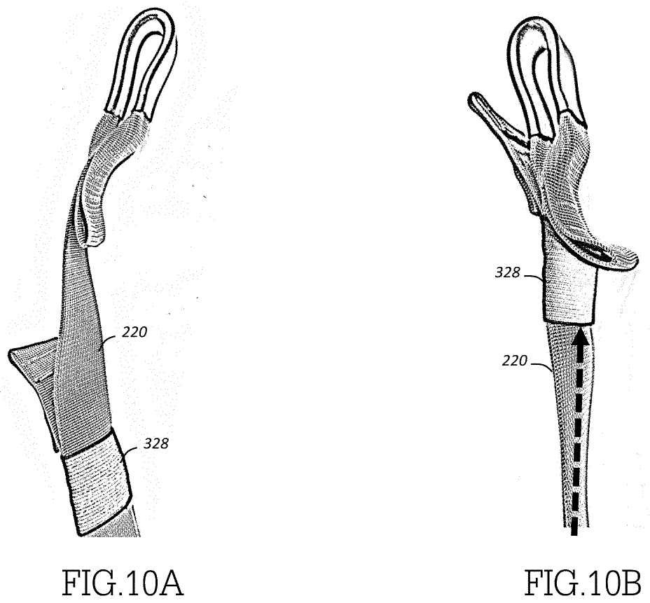

Referring to FIGS. 10A and 10B, a sleeve 328 can be positioned around each band 220, proximate a second end of the band 220. The sleeve 328 is formed from an elastic material such as silicone. The sleeve 328 encloses an end portion of the band 220 proximate the second end, and an adjacent portion of the band 220 onto which the end portion is folded and overlayed. The sleeve 328 thus restrains the end portion of the band 220 in relation to the adjacent portion of the band 220. The sleeve 328 is secured to the underlying end portion of the band 220 by a suitable means such as stitching. The sleeve 328 is not secured to the adjacent or overlapping portion of the band 220. Thus, when adjusting the effective length of the band 220, user can grasp the second end of the band 220 and move the second end and the attached sleeve 328 up or down the adjacent portion of the band 220 as depicted in FIG. 10B, to increase or decrease the extent to which the band 220 is looped.

As can be seen in FIG. 10B, the stitching that secures the overlying portions of the first end of the band 220 to form the loop 230 acts as a stop for the sleeve 328 as the sleeve 328 approaches the first end of the band 220. This feature helps to prevent the portion of the band 220 that is not fixed to the sleeve 328 from disengaging from the sleeve 328. Alternative embodiments can be configured without the sleeve 328.

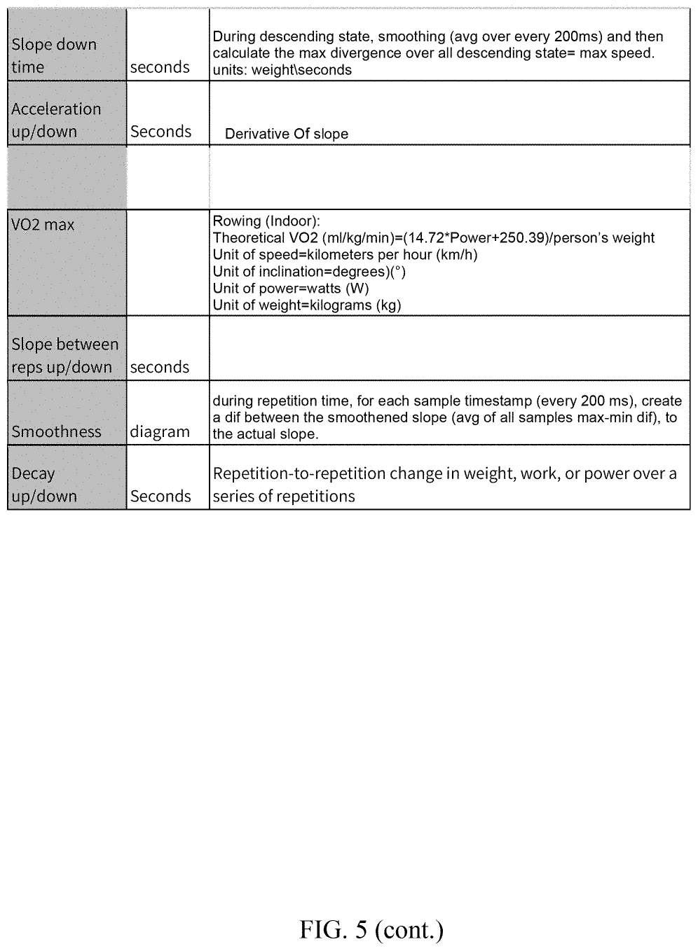

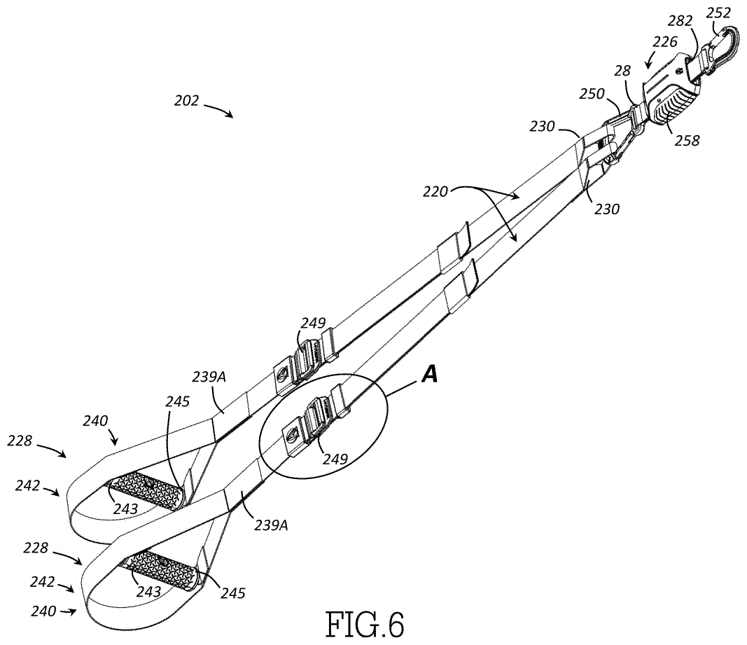

Referring to FIGS. 8A and 8B, the force sensor 226 comprises a load cell 256, a housing 258, a first attachment means, and a second attachment means. The first attachment means can be, for example, a spring-loaded carabiner 250. The carabiner 250 is attached to a first end of the load cell 256 by way of a strap 281. The strap 281 is formed from an inelastic, flexible material such as nylon. The strap 281 can be formed from an elastic material in alternative embodiments. The strap 281 is configured so that a loop is formed at each end of the strap 281; and the overlapping portions of the strap 281 between the loops are joined by stitching or other suitable means. A first of the loops engages the carabiner 250. The second loop engages the load cell 256 in the manner discussed below.

The carabiner 250 is configured to engage the loop 230 in each band 220, so that the user-generated force imparted to the bands 220 by way of the handles 228 can be transmitted from the bands 220 and to the force sensor 226 by way of the carabiner 250 and the strap 281. The carabiner 250 allows the user to connect and re-connect the bands 220 to the force sensor 226. The suspension training device 202 can be used with both of the bands 220 connected to the force sensor 220. The user also has the option to use the suspension training device 202 while only one of the bands 220 connected to the force sensor.

The second attachment means, can be, for example, a spring-loaded D-ring 252. The D-ring 252 is attached to a second end of the load cell 256 by way of a strap 282. The strap 282 is formed from an inelastic, flexible material such as nylon. The strap 282 can be formed from an elastic material in alternative embodiments. The strap 282 is configured so that a loop is formed at each end of the strap 282; and the overlapping portions of the strap 282 between the loops are joined by stitching or other suitable means. A first of the loops engages the load cell 256 in the manner discussed below.

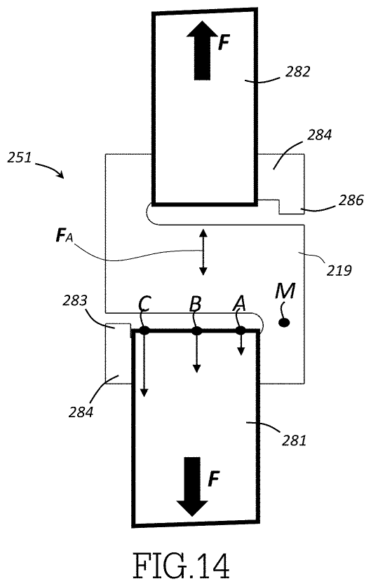

As can be seen in FIG. 13, the second loop of the strap 282 can engage a stationary anchoring point for the suspension training device 202, by way of the D-ring 252. The anchoring point can be, for example, a hook or other attachment means (not shown) that is securely attached to or mounted on a stationary structure such as a wall. The user-generated force is transmitted from the load cell 256 and to the anchoring point by way of the strap 282 and the D-ring 252. The D-ring 252 allows the user to connect and re-connect the force sensor 226 (and the suspension training device 202) to the anchoring point.

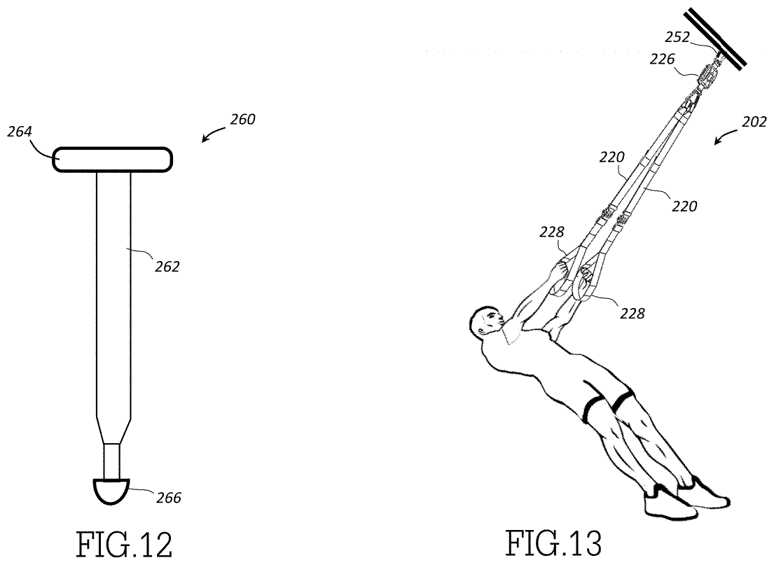

Alternatively, the suspension training device 202 can include an anchor 260 for attaching the suspension training device 202 to a stationary structure. The anchor 260 is depicted in FIG. 12. The anchor 260 includes a strap 262, and a relatively large restraining portion 264 securely attached to a first end of the strap 262 by a suitable means such as stitching. An attachment means, such as a spring-loaded D-ring 266, is attached to a second end of the strap 262. The D-ring 266 engages the D-ring 252 of the force sensor 226, to couple the force sensor 226 (and the suspension training device 202) to the anchor 260.

The restraining portion 264 and the first end of the strap 262 can be positioned on one side of a door or other movable structure, and the D-ring 266 and the second end of the strap 262 can be positioned on the other side of the door. Once the door is closed, the strap 262 extends between the outer periphery of the door and the adjacent surface of the door frame. Interference between the restraining portion 264 and the adjacent surfaces of the door and the door frame causes the restraining portion 264 to restrain the strap 262 as the second end of the strap 262 is pulled away from the restraining portion 264 and the door as the user exerts tension on the strap 262 by way of the handles 228 and the bands 220.

A user can generate a resistive force by pulling the handles 228. For example, the user can lean away from the anchoring point of the suspension training device 202 while facing the anchoring point, as shown in FIG. 13, so that the user raises his or her body as the user pulls on the handles 228. The force exerted by the user on the handles 228 causes the attached bands 220 to become tensioned. The force is transmitted to the structure to which the suspension training device 202 is anchored, by way of the force sensor 226. The anchoring structure generates a reactive force that is transmitted to the user by way of the force sensor 226, which in turn activates one or more muscle groups of the user to work against the reactive force as the user continues to pull the handles 228. The specific muscle groups activated during a particular exercise depends on the type of exercise being performed, the position and orientation of the user in relation to the handles 228, whether the user is engaging the handles 228 with the user's hands or feet, etc.

The force sensor 226 measures the resistance felt by the user as the use pulls the handles 228. Referring to FIGS. 8A and 8B, the force sensor 226 comprises the load cell 256, and a housing 258 that covers the load cell 256. The load cell 256 can be, for example, an S-beam load cell comprising an S-shaped beam 257.

The beam 257 is positioned within the housing 258. The beam 257 includes a central portion 279, a first arm 280 that adjoins the central portion; and a second arm 284. The second arm 284 adjoins the central portion 279, on an opposite side and opposite end of the central portion 279 from the first arm 280.

The first arm 280 is disposed within the second loop in the strap 281, thereby connecting the load cell 256 to the carabiner 250. The second arm 284 is disposed within the first loop of the strap 282, thereby connecting the load cell 256 to the D-ring 252. The straps 281, 282 extend through respective openings in the housing 258. In the alternative embodiment depicted in FIGS. 15 and 16, the loop 230 can be placed directly over the first arm 280 to form a permanent connection between the band 220 and the force sensor 226; or the loop 230 can be removably connected to the first arm 280 by way of the carabiner 250 and strap 281.

Referring again to FIGS. 8A and 8B, the first arm 280 has a lip 283 formed on a freestanding end thereof. The lip 283 helps to retain the second loop of the strap 281 on the first arm 280, by discouraging the second loop from sliding off of the freestanding end of the first arm 280. The second arm 284 likewise has a lip 286 formed on a freestanding end thereof. The lip 286 helps to retain the first loop of the strap 282 on the second arm 284, by discouraging the first loop from sliding off the freestanding end of the second arm 284. Alternative embodiments of the load cell 256 can be formed without the lips 283, 286; and the straps 281, 282 can be retained on the respective first and second arms 280, 284 by the housing 258, or by other suitable means.

As can be seen in FIGS. 8A and 8B, the first and second arms 280, 284 are configured so that the loops of the first and second bands 281, 282 fit over the respective first and second arms 280, 284 without being bunched, twisted, or otherwise deformed. Such deformation, if allowed to occur, could affect the direction and magnitude of the forces transmitted to and from the force sensor 226, which in turn could adversely affect the accuracy of the force readings generated by the force senor 226. Deformation of the first strap 281 is avoided by configuring the first arm 280 so that the distance between the lip 283 on the first arm 280, and the non-freestanding end of the first arm 280 is about equal to the width, or side to side dimension, of the strap 281. This distance is denoted in FIG. 8B by the reference character “d1.” Deformation of the second strap 282 likewise is avoided by configuring the second arm 284 so that the distance between the lip 286 on the second arm 284, and the non-freestanding end of the second arm 284 is about equal to the width of the strap 282. This distance is denoted in FIG. 8B by the reference character “d2.”

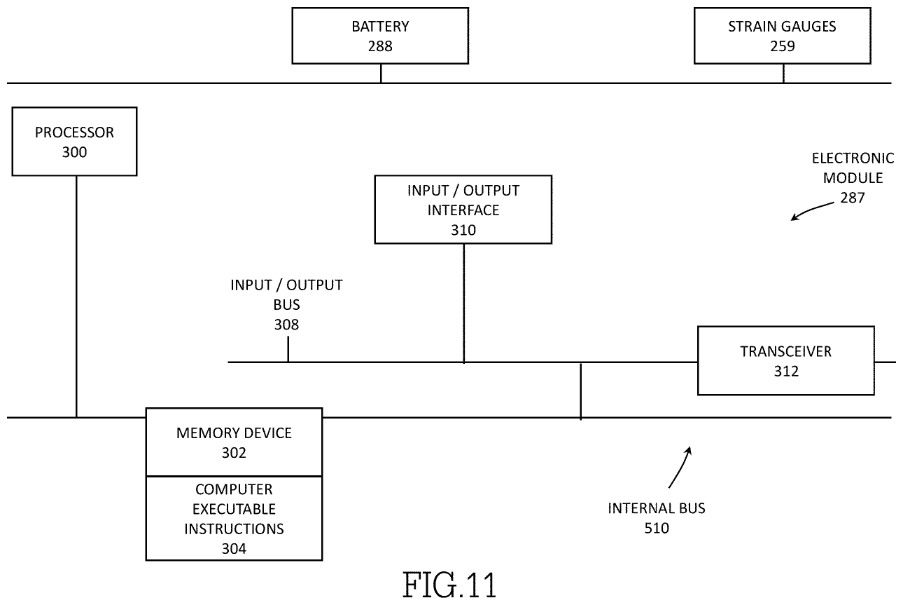

The load cell 256 includes one or more internally-located strain gauges 259, depicted in FIG. 11. The strain gauges 259 generate a response related to the external forces exerted on the load cell 256 by way of the bands 281, 282.

The force sensor 226 also includes a computing device in the form of an electronics module 287 communicatively coupled to the strain gauges 259, and a battery 288 that provides power to the electronics module 287. The electronics module 287 provides an excitation voltage to the strain gauges 259, and is configured to process the electrical output of the strain gauges 259. The electronics module 287 and the battery 288 are shown in FIGS. 8A and 11.

Referring to FIG. 11, the electronics module 287 comprises a processor 300, such as a microprocessor; a memory device 302 communicatively coupled to the processor 300 via an internal bus 303; and computer-executable instructions 304 stored on the memory device 302 and executable by the processor 300. The electronics module 287 also comprises an input-output bus 308; an input-output interface 310 communicatively coupled to the processor 300 by way of the input-output bus 308, and a transceiver 312 communicatively to the input-output interface 310. The computer-executable instructions 304 are configured so that the computer-executable instructions 304, when executed by the processor 300, cause the electronics module 287 to carry out the various logical operations described herein.

The above details of the electronics module 287 are presented for illustrative purposes only. The electronics module 287 has components in addition to those described above and can have an internal architecture other than that described above.

The electronics module 287 is configured to generate force measurements representing the force being transmitted through the force senor 226. The force measurements are based on the response of the strain gauges 259 to the external loads applied to the load cell 256 via the first and second bands 281, 282; and calibration data stored in the memory 302. The force measurements correspond to the loads being applied by the user to the handles 228. The electronics module 287 continually transmits the force readings to the user interface of the system 200, i.e., the smartphone 16, along with a unique identifier associated with the force sensor 226. The force measurements are transmitted wirelessly to the smartphone 16 by the transceiver 312, using a suitable wireless means such as BLUETOOTH.

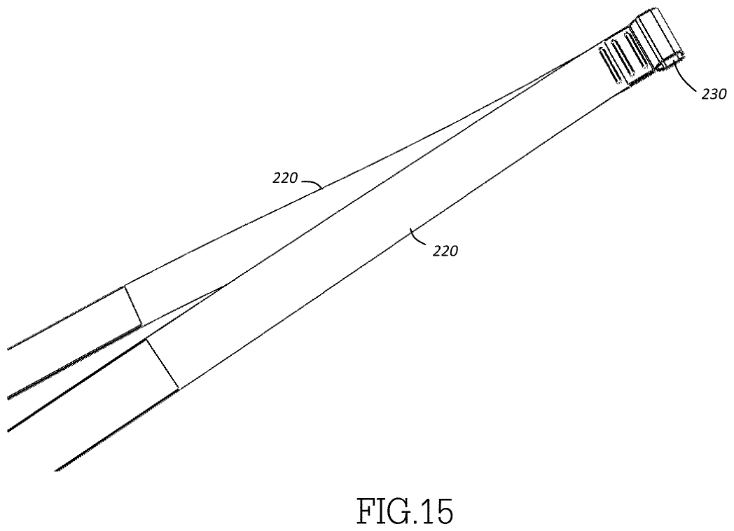

Because one end of each of the first and second arms 280, 284 is freestanding, the deflections of the first and second arms 280, 284 in response to the forces applied to the force sensor 226 are not uniform across the respective lengths of the first and second arms 280, 284. For example, FIG. 14 diagrammatically depicts the deflection of the first arm 280 in response to a force applied thereto via the strap 281. As can be seen in FIG. 14, point C is located at a greater distance from the point of restraint of the first arm 280, which is located proximate point M. Thus, point C is located on a longer moment arm than points A and B in relation to the point of restraint, and the toque resulting from the force applied to the first arm 280 causes point C to deflect by a larger distance than points A and B. Similarly, because point B is located on a longer moment arm than point A in relation to the point of restraint, the toque resulting from the force applied to the first arm 280 causes point B to deflect by a larger distance than point A. Although not illustrated in FIG. 14, the second arm 284 behaves in a similar manner in response to an applied force

Because the deflection of the first and second arms 280, 284 is non-uniform along the respective lengths of the first and second arms 280, 284, the respective force vectors acting on the first and second arms 280, 284 each have a component that is not aligned with the force-measurement axis of the load cell 256. The force-measurement axis is denoted in FIG. 14 by the character “FA.” This potential source of error in the force measurements generated by the force sensor 226 is addressed by calibrating the force sensor 226 prior to use. In particular, a series of known forces are applied to the force sensor 226, the response of the force sensor 226 is measured and recorded, and a calibration curve or other calibration data is generated based on a correlation between the known applied loads and the response of the force sensor 226 thereto. The calibration data can be stored on the memory device 302 of the electronics module 287 and can be accessed and applied by the processor 300 to calculate the forces being applied to the force sensor 226 based on the responses of the strain gauges 259 of the load cell 256.

In alternative embodiments, the calibration data can be stored on, and the force calculations can be performed by a computing device other than the electronics module 257, such as the smartphone 16.

The smartphone 16 and the server 18 process, store, display, and otherwise manipulate the force measurements as described above in relation to the system 10. The smartphone 16 and the server 18 likewise can guide the user through a particular exercise program and can adjust the difficulty of the program based on the user's performance and preferences, as also discussed above in relation to the system 10.

As noted above, the user can exercise using the suspension training device 202 by grasping the handles 228 while facing the anchoring point of the suspension training device 202, leaning away from the anchoring point of the system 200, and pulling the handles 228 so that the user partially lifts his or her body. The user's own body weight thus provides the resistance that activates and exercises the targeted muscle group of the user. Also, the position of the user affects the difficulty level of this particular exercise. Specifically, the user can angle his or her body away from the anchoring point for the system 200, as shown in FIG. 13. Increasing the angle at which the user leans rearward, away from the anchoring point, increases the resistance that the user experiences when pulling on the handles 228, because the user will be lifting a higher percentage of the user's body weight when the user leans further back. Conversely, the user can reduce the resistance felt by the user by assuming a more upright position. Thus, the difficulty level of the exercise program can be varied by positioning the user closer to or further away for the anchoring point of the suspension training device 202. In this particular type of exercise, once the user has found the proper position, or distance from the anchoring point, that produces the desired amount of resistance, the user remains in that position. The resistance felt by the user varies during the subsequent repetitions as the user raises and lowers his or her body during each repetition. One possible variation of this exercise can be performed with the user facing away from the anchoring point of the suspension training device 202, while also leaning away from the anchoring point. In another possible variation, the user can grasp both handles 228 with one hand and exert force on the bands 220 with that one hand.

As discussed above in relation to the system 10, the system 200 can guide the user through a particular exercise program. Specifically, the system 200 can instruct the user, via the smartphone 16, to exert a particular force on the handles 228 based on the targeted difficulty level of the exercise program being performed. The system 200 can determine the targeted difficulty level based on the user's previous performance, and/or an input by the user regarding the difficultly level preferred by the user for that particular exercise program, as discussed above in relation to the system 10. The desired force to be exerted by the user can be, for example, a percentage of the user's body weight. If the force, as sensed by the force sensor 226, is lower than the targeted force, the system 200 can instruct the user to take one or more steps forward, i.e., toward the anchoring point, to cause the user to lean further back. The increased angle of the user in relation to the ground will cause the user to lift a higher percentage of his or her body weight during each repetition, which in turn will cause the force exerted by the user, and the difficulty level of the exercise program, to increase. Conversely, if the sensed force is too high, the system 200 can instruct the user to take one or more steps rearward, to decrease the extent to which the user leans back. The decreased angle of the user in relation to the ground will cause the user to lift a lower percentage of his or her body weight during each repetition, which in turn will cause the force exerted by the user, and the difficulty level of the exercise program, to decrease. The resistance readings provided by the system 200 thus can help the user to understand how much weight the user is lifting during each repetition.

The angle of the user in relation to the ground also can be adjusted by varying the length of the bands 220. The smartphone 16 and/or the server 18 can be configured to provide the user with guidance regarding where the user should stand in relation to the anchoring point, and the length to which the bands 220 should be adjusted, to produce the resistance called for in a particular exercise program. In alternative embodiments, the bands 220 can include multiple markings that user can use to adjust the length of the bands 220 in accordance with guidance provided by the system 200 via the smartphone 16. Based on the resulting force readings displayed on the smart phone 16, the user then can make final adjustments to the resistance level by moving closer to or further from the anchoring point, and/or by changing the length of the bands 220. The force readings can be displayed as the actual force measured by the force sensor 226, and/or as a percentage of the user's body weight.

As discussed above in relation to the system 10, the system 200 can guide the user through the exercise program, providing the user with visual and audible cues via the smartphone 16. For example, the system 200 can guide the user through a specific number of repetitions at a specific force level, over a specific time period, and with a specific time between repetitions. Also, the system 200 can be configured to provide the user with real-time feedback via the smartphone 16. For example, the smartphone 16 can provide the user with a visual or audible cue after every repetition, or after a predetermined number of repetitions, indicating whether the user is or is not meeting the targeted force, the targeted number of repetitions, the target time between repetitions, etc.

Some types of exercises cannot be tracked accurately by repetitions. Such exercises include, for example, static exercises; combos; and exercises where the movement of the user does not substantially change the tension in the bands 220, e.g., plank pushups where the user's arms move while the user's legs remain substantially static. These types of exercise programs can be focused on time and weight (force), and not repetitions.

The smartphone 16 and/or the server 18 are configured to recognize the different types of exercises, and to tailor the recommended resistance levels and repetitions to 10 to each particular exercise type, as well as to the particular user.

For example, the user can place his or her feet in the foot cradle provided by the handles 228 while facing and lying parallel to the floor and extending his or her arms. The user can bend at the waist to raise the user's torso; the user then can lower his or her waist to return to the starting position. The user can be prompted by the smartphone 16 to repeat these motions. In this type of exercise, the user's position on the bands 220 remains substantially the same, and any changes in the resistance felt by the user are minor.

In another type of exercise that can be performed using the system 200, the user can place his or her feet in the foot cradle provided by the handles 228 while facing and lying parallel to the floor and extending his or her arms. The user then can perform push-ups with the user's legs suspended in this manner. In this type of exercise, the user's position on the bands 220 remains substantially the same, and the movement is not related to the bands 220.

In another type of exercise that can be performed using the system 200, the user can place his or her feet in the foot cradle provided by the handles 228 while facing and lying parallel to the floor and extending his or her arms. The user can remain in this modified plank position. This type of exercise is a static exercise, with no repetitive movement.

In another type of exercise that can be performed using the system 200, the user can grasp the handle 228 while standing, and facing the anchoring point of the suspension training device 202 while leaning slightly away from the anchoring point. The user then can perform squats while holding the handles 228. In this type of exercise, the user's position on the bands 220 remains substantially the same, and the change in resistance through the movement is minimal

The system 200 (and the system 10) can be further configured to guide the user through a stretching program at the conclusion of the exercise program.

The force sensor 226 is not limited to use as part of the system 200. The force sensor 226 can be used in other types of training systems, including training systems that include resistance bands, i.e., bands that elastically deflect in response to being placed in tension.

We claim:

1. An exercise system, comprising:
at least one band;
at least one handle configured to be coupled to the at least one band;
a force sensor comprising:
a load cell comprising: a beam configured to be coupled to the at least one band, and to an anchoring point; and at least one strain gauge mounted on the beam; and
a first computing device communicatively coupled to the strain gauge and comprising a processor configured to determine a force acting on the force sensor based on an output of the strain gauge; and
a second computing device communicatively coupled the first computing device and configured to display information relating to an exercise session performed on the system by a user.

2. The system of claim 1, wherein the beam is a substantially S-shaped beam comprising a first arm configured to be coupled to the at least one band; and a second arm configured to be coupled to the anchoring point.

3. The system of claim 2, wherein the force sensor further comprises:
a first strap coupled to the first arm of the beam and configured to be coupled to the at least one band; and
a second strap coupled to the second arm and configured to be coupled to the anchoring point.

4. The system of claim 3, wherein:
the first strap has a first loop formed therein;
the first strap is connected to the first arm of the beam by way of the first loop;
the second strap has a second loop formed therein; and
the second strap is connected to the second arm of the beam by way of the second loop.

5. The system of claim 1, wherein the at least one band is an inelastic band.

6. The system of claim 3, wherein:
the beam of the load cell further comprises a first lip located at a freestanding end of the first arm, and a second lip located at a freestanding end of the second arm;
the first lip is configured to retain the first strap on the first beam; and
the second lip is configured to retain the second strap on the second beam.

7. The system of claim 6, wherein:
a distance between the first lip and a non-freestanding end of the first arm is about equal to a width of the first strap; and
a distance between the second lip and a non-freestanding end of the second arm is about equal to a width of the second strap.

8. The system of claim 1, wherein:
the first computing device comprises a memory having calibration data for the load cell stored therein; and
the processor is further configured to determine the force acting on the force sensor based on the output of the strain gauge and the calibration data.

9. The system of claim 1, further comprising a buckle configured to connect the at least one handle to the at least one band; and a restraint, wherein the restraint comprises:
a first sleeve configured to receive overlapping portions of the at least one band;
a second sleeve configured to receive a portion of a strap of the at least one handle; and
a tether connected to the first and second sleeve and configured to straddle the buckle.

10. The system of claim 9, wherein the restraint is restricted from substantial relative movement in lengthwise directions of the strap and the at least one band by interference between the first sleeve and the buckle, interference between the second sleeve and the buckle, and mutual restraint of the first and second sleeves by way of the tether.

11. The system of claim 1, further comprising a sleeve configured to receive overlapping portions of the at least one band, wherein the sleeve is fixed to only one of the overlapping portions of the at least one band.

12. The system of claim 3, wherein the at least one band is a first band; and the system further comprises a second band.

13. The system of claim 12, wherein:
respective end portions the first and second bands overlap, are secured to each other, and define a loop; and
the first and second bands are configured to be connected to the first arm of the beam by way of the loop.

14. The system of claim 1, wherein the second computing device is further configured to display, on a real-time or near real-time basis, the force acting on the force sensor.

15. The system of claim 1, wherein the second computing device is further configured to calculate and display a percentage of the exercise session that has been completed by the user.

16. The system of claim 1, wherein the second computing device is further configured to calculate target values for a force to be applied to the at least one handle by the user, based on the performance of the user during the exercise session or during a previous exercise session.

17. The system of claim 1, wherein the second computing device is further configured to calculate and display target values for a rate and a number of repetitive applications of a force to be applied to the at least one handle by the user, and to display an actual rate and an actual number of repetitive applications of the force applied by the user to the at least one handle.

18. The system of claim 1, wherein the second computing device is further configured to recommend to a user a difficulty level of an exercise session based on performance of the user during one or more prior exercise sessions.

19. The system of claim 1, wherein the second computing device is a smartphone.

20. An exercise system, comprising:
a force sensor comprising:
a load cell comprising: a beam configured to be coupled to at least one band, and to an anchoring point; and at least one strain gauge mounted on the beam; and
a first computing device communicatively coupled to the strain gauge and comprising a processor configured to determine a force acting on the force sensor based on an output of the strain gauge; and
a second computing device communicatively coupled the first computing device and configured to display information relating to an exercise session performed on the system by a user.

21. A force sensor, comprising:
a load cell comprising: a substantially S-shaped beam comprising a first and a second arm; and at least one strain gauge mounted on the beam;
a first computing device communicatively coupled to the strain gauge and comprising a processor configured to determine a force acting on the force sensor based on an output of the strain gauge;
a first strap having a first loop formed therein and configured to be coupled to a band, the first strap being connected to the first arm by way of the first loop; and
a second strap having a second loop formed therein and configured to be coupled to an anchoring point, the second strap being connected to the second arm by way of the second loop.
